(19) United States
(12) Patent Application Publication
(10) Pub. No.: US 2008/0294175 A1
(43) Pub. Date: Nov. 27, 2008

(54) LEFT ATRIAL APPENDAGE CLOSURE

(75) Inventors: Earl Bardsley, Newton, MN (US); Jianlu Ma, Maple Grove, MN (US); Anthony Huynh, Mahtomedi, MN (US)

Correspondence Address: HAMRE, SCHUMANN, MUELLER & LARSON, P.C., P.O. BOX 2902, MINNEAPOLIS, MN, 55402-0902, US

(73) Assignee: EPITEK, INC., Bloomington, MN (US)

(21) Appl. No.: 12/183,345

(22) Filed: Jul. 31, 2008

Related U.S. Application Data
(63) Continuation-in-part of application No. 12/119,008, filed on May 12, 2008.
(60) Provisional application No. 60/939,210, filed on May 21, 2007.

(51) Int. Cl. A61B 17/26; A61B 17/08

(52) U.S. Cl. 606/113; 606/213

(57) ABSTRACT

A medical device is disclosed for tissue, body lumen and/or cavity closure inside a body of a patient. In one particular application, the medical device can be used for minimally invasive access and closure of a left atrial appendage of the heart. The medical device generally includes a tool used for grasping the appendage, a closure member, and at least one tool to deploy, control, and position the closure member for closing the appendage. The device can also include an expander tool for expanding the working area around the left atrial appendage to improve visibility during the procedure. In other embodiments, the medical device may include other tools, for example an imaging tool for viewing the target area and/or other tools that are considered useful in a left atrial appendage closure procedure.

### CROSS-REFERENCE TO RELATED APPLICATIONS

The present application is a continuation-in-part application of U.S. application Ser. No. 12/119,008 entitled “LEFT ATRIAL APPENDAGE CLOSURE” filed on May 12, 2008, which claims the benefit of U.S. Provisional Patent Application Ser. No. 60/939,210 entitled “LEFT ATRIAL APPENDAGE CLOSURE,” filed on May 21, 2007, and which both are herewith incorporated by reference in their entirety.

### FIELD

This disclosure relates to methods and devices useful for a variety of medical procedures for tissue, body lumen and/or cavity closure, for example minimally invasive access and closure of a left atrial appendage of the heart.

### BACKGROUND

Medical devices for implementing medical procedures for tissue, body lumen and/or cavity closure are known, including those for accessing and closing an appendage. Typically, these devices have employed various tools, which have included tools to access an anatomical area where tissue, lumen or cavity resides, tools to grasp the tissue, lumen or cavity, tools to deploy a closure suture, tools to close the tissue, lumen or cavity with the closure suture, and tools to release the closure suture. As one particular example, such devices have been used for access and closure of a left atrial appendage.

Atrial fibrillation is a common cardiac rhythm disorder affecting a population of approximately 2.5 million patients in the United States alone. Atrial fibrillation results from a number of different causes and is characterized by a rapid chaotic heart beat. In addition to the risks associated with a disordered heart beat, patients with atrial fibrillation also have an increased risk of stroke. It has been estimated that approximately 75,000 atrial fibrillation patients each year suffer a stroke related to that condition. It appears that strokes in these patients result from emboli, many of which may originate from the left atrial appendage of the heart. The irregular heart beat causes blood to pool in the left atrial appendage, allowing clots to accumulate over time. From time to time, a clot may dislodge from the left atrial appendage and may enter the cranial circulation causing a stroke, the coronary circulation causing a myocardial infarction, the peripheral circulation causing limb ischemia, as well as other vascular beds.

Significant efforts have been made to reduce the risk of stroke in patients suffering from atrial fibrillation. Most commonly, those patients are treated with blood thinning agents, such as coumadin, to reduce the risk of clot formation. While such treatment can significantly reduce the risk of stroke, it also increases the risk of bleeding and for that reason is inappropriate for many atrial fibrillation patients.

As an alternative to drug therapy, surgical procedures for closing the left atrial appendage have been proposed. Most commonly, the left atrial appendage has been closed or removed in open surgical procedures, typically where the heart has been stopped and the chest opened through the sternum. Because of the significant risk and trauma of such procedures, left atrial appendage closure or removal occurs almost exclusively when the patient's chest is opened for other procedures, such as coronary artery bypass or valve surgery.

Recently, sub-xiphoid approaches to left atrial appendage closure have been proposed. See, for example, U.S. Pat. No. 6,488,689 and U.S. Patent Application Publication 2007/0027456. In these approaches, a percutaneous penetration is first made beneath the rib cage, preferably between the xiphoid and adjacent costal cartilage, and an atrial appendage closure tool advanced through the penetration, over the epicardial surface (in the pericardial space) to reach a location adjacent to the exterior of the left atrial appendage. The appendage is then closed using a suitable closure mechanism, for example a closure loop.

Despite existing technology, further improvements relating to accessing and closing a left atrial appendage are desirable.

### SUMMARY

An improved medical device is described that can be used in medical procedures for tissue, body lumen and/or cavity closure. In one specific application described herein, the medical device can be used for minimally invasive access and closure of a left atrial appendage of the heart. However, the medical device and its components can be used for other tissue, body lumen and/or cavity closure procedures and other medical procedures.

When used for minimally invasive access and closure of a left atrial appendage of the heart, the medical device generally includes a tool used for grasping the appendage, a closure member, and at least one tool to deploy, control, and position the closure member for closing the appendage. The device can also include an expander tool for expanding the working area around the left atrial appendage to improve visibility during the procedure. In other embodiments, the medical device may include other tools, for example an imaging tool for viewing the target area and/or other tools that are considered useful in a left atrial appendage closure procedure.

In one embodiment, at least the grasping tool, the closure member, the imaging tool, and the tool to deploy, control, and position the closure member are part of the same sub-assembly, referred to herein as the closure sub-assembly, while the expander tool, which forms part of an expander sub-assembly, is separate from the closure sub-assembly. The sub-assemblies together form the medical device, and are configured to be used together during a closure procedure.

### DRAWINGS

FIG. 1 shows a model of a heart with the left atrial appendage and one embodiment of a medical device for closing the left atrial appendage.

FIG. 2 is one embodiment of a closure sub-assembly.

FIG. 3 is a side view in partial section of the tip of the closure sub-assembly with the tools retracted within a lumen tube of the sub-assembly.

FIG. 4 is a side view in partial section of the tip of the closure sub-assembly with some of the individual tools extended from the tip.

FIG. 5A is a perspective view of one embodiment of a multi-lumen tube of the closure sub-assembly.

FIG. 5B is an embodiment of an endoscope extending through the multi-lumen tube of FIG. 5A.

FIG. 6A is one embodiment of a snare mechanism usable with the closure sub-assembly.

FIG. 6B is a cross-sectional view of the snare mechanism.

FIG. 7 shows details of one embodiment of a knot pusher and suture trimmer for cinching and cutting the suture of the snare mechanism.

FIG. 8 shows one embodiment of an access sheath together with one embodiment of an expander sub-assembly.

FIG. 9 shows the tip of the expander sub-assembly with one embodiment of an expander tool covered by a loading sheath.

FIG. 10 illustrates the expander tool in an expanded state extending from the end of the access sheath.

FIG. 11 illustrates details of the expander sub-assembly.

FIG. 12 is a view of the expander tool in a flat, unrolled condition.

FIGS. 13A-C are cross-sectional views of the expander sub-assembly in operation.

FIG. 14 illustrates an alternate embodiment of a closure sub-assembly.

FIGS. 15A and 15B illustrate different embodiments of snare retention/release mechanisms.

FIGS. 16A and 16B illustrate further embodiments of snare retention/release mechanisms.

FIG. 17 illustrate another embodiment of a snare retention/release mechanism.

FIG. 18 illustrates an alternative embodiment of an expander tool.

FIG. 19 shows alternative details for a tube with a closure member support which is part of a mechanism used to advance and position a closure member.

FIG. 20A-B show additional views of the tube of FIG. 19.

FIG. 21 shows yet another alternative of details for a closure member support which is part of a mechanism used to advance and position a closure member.

FIGS. 22A-C shows additional views of the closure member support of FIG. 21.

FIG. 23 shows an alternative embodiment of a configuration for a closure member.

FIGS. 24A-B show an alternative embodiment of device actuators.

FIG. 25 shows an alternative embodiment of a constricting tool.

FIG. 26 shows an alternative embodiment of a tool to deploy, control, and position a closure member of a constricting tool.

FIGS. 27-28 show an alternative for deploying, controlling, and positioning a closure member.

FIG. 29 shows an alternative embodiment for deploying and cutting a single suture pull leg.

FIG. 30A shows another embodiment of a tool to deploy, control, and position a closure member of a constricting tool.

FIG. 30B shows a sectional view of the tool to deploy, control, and position a closure member of a constricting tool taken from line A-A of FIG. 30.

FIG. 31 shows the tool to deploy, control, and position a closure member of a constricting tool of FIG. 30A and in a drawn down position.

FIG. 32 shows an alternative embodiment of a constricting or closure tool.

FIG. 33 shows the constricting tool of FIG. 32 and in a retracted position.

FIG. 34 shows the constricting tool of FIG. 32 and in a drawn down position.

FIG. 35 shows the constricting tool of FIG. 32 and in a joined position.

### DETAILED DESCRIPTION

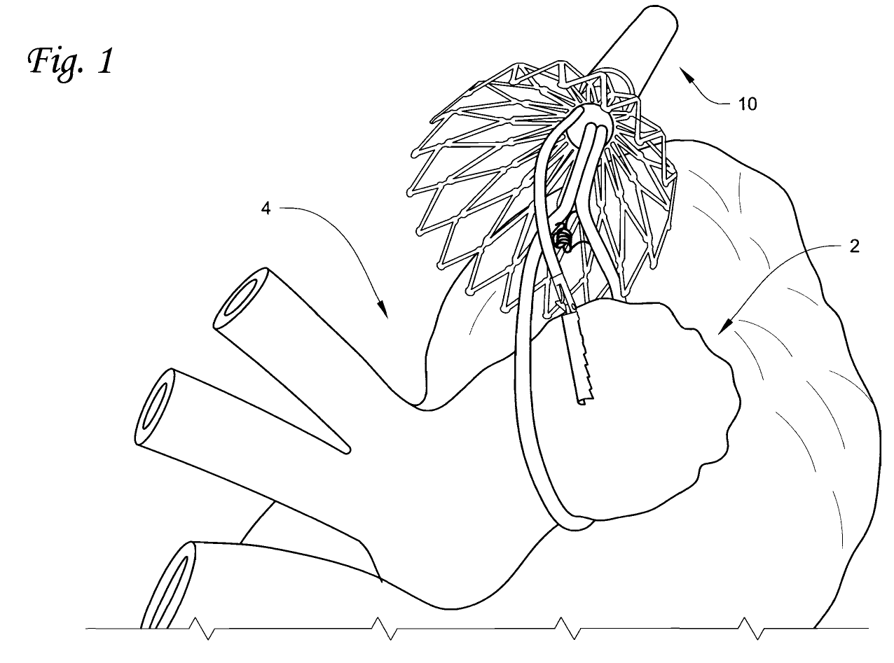

A medical device 10 that can be used for minimally invasive access and closure of a left atrial appendage 2 of a human heart 4 is illustrated in FIG. 1. The device 10 is particularly configured for use in a sub-xiphoid procedure, but could be used in other types of procedures as well. While the following description will describe the device 10 with respect to left atrial appendage closure applications, it is to be understood that the device 10 and individual components of the device 10 discussed below are not necessarily limited to left atrial appendage closure applications. The medical device 10 can be used in a number of differing medical applications, including applications where one or more of non-traumatic grasping, manipulation, closure, and inspection of anatomical tissue is required, for example tissue, body lumen and/or cavity closure.

The device 10 generally includes a closure sub-assembly 5, an expander sub-assembly 6, and an introducer sheath 7. The sub-assemblies 5, 6 and the sheath 7 together form the medical device, and are configured to be used together during a closure procedure.

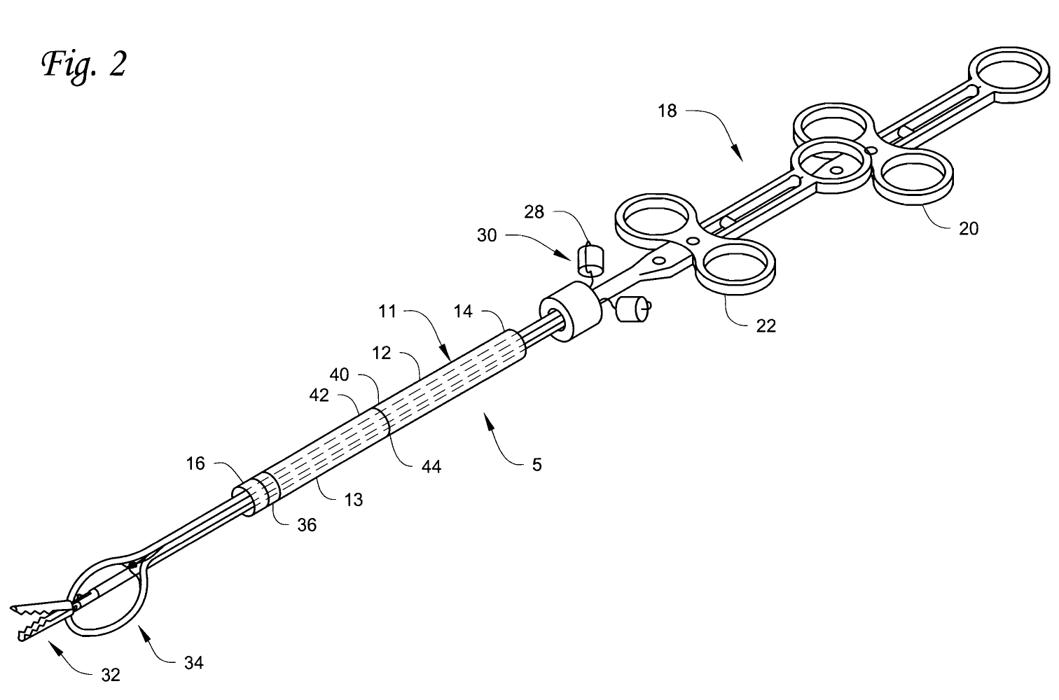

With reference to FIG. 2, the closure sub-assembly 5 is illustrated. The sub-assembly 5 includes a tube 11 composed of a multi-lumen tube 12 having a proximal end 14, and a lumen tube 13, which may be a single or multi-lumen tube as further described below, that is connected to an end of the multi-lumen tube 12, with the lumen tube 13 having a distal end 16. A number of tools, the purpose, construction and function of which are described below, extend through the multi-lumen tube 12 and the lumen tube 13. At the proximal end 14, a number of actuators 18 are provided that are connected to the tools for manipulating the tools. The actuators 18 can include, for example, an actuator 20 for actuating a grasping tool, and an actuator 22 for actuating a closure member. A viewing scope (not shown) connected to a camera can also be disposed at the proximal end 14. In addition, a free end 28 of a pull suture 30 can extend from the proximal end 14 and can act as an actuator for contracting the closure member.

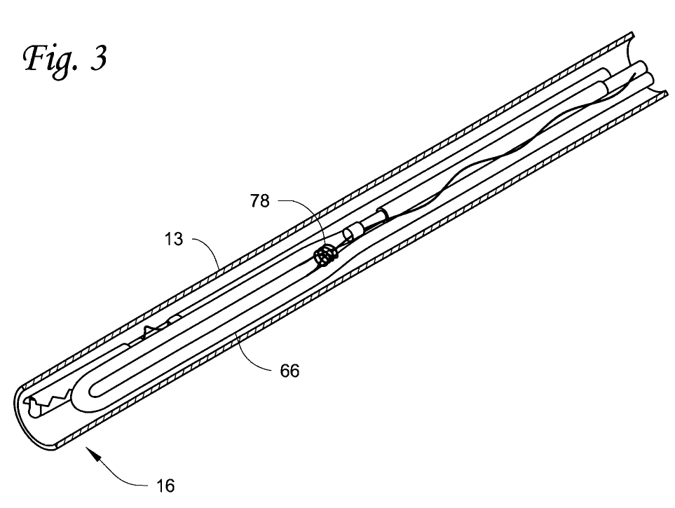

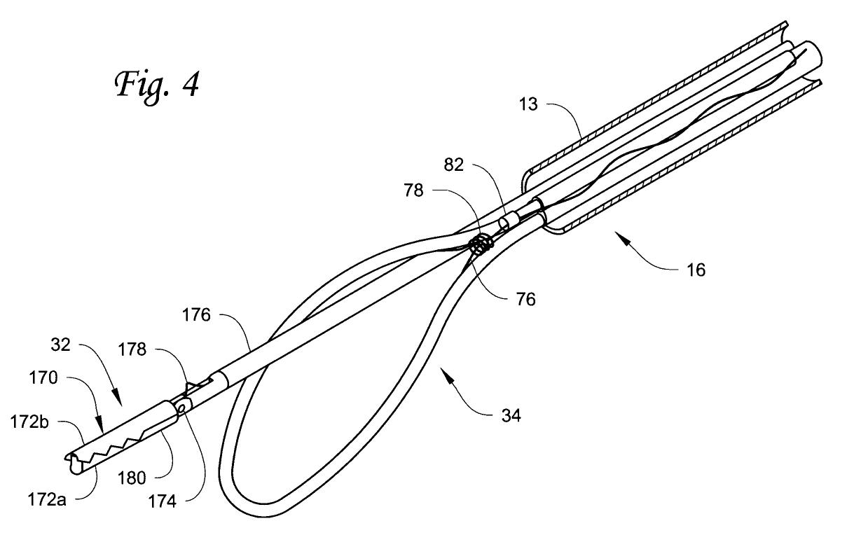

As will be described below, many of the tools of the sub-assembly 5 are mounted within the tubes 12, 13 to permit independent operation, including axial movement relative to the tubes 12, 13, actuated by the respective actuators. FIG. 3 illustrates the distal end 16 of the tube 13 with the tools fully retracted, or in a stowed position, within the end of the tube 13. FIGS. 2 and 4 illustrate a grasping tool 32 and a constricting tool 34 advanced axially by the respective actuators 20 and 22 relative to the tube 13 so that they extend beyond the distal end 16 (i.e. a deployed position).

A ring 36 is connected near the end 16 of the tube 13, as shown in FIG. 2. The ring 36 is used for visualization, for example using fluoroscopy, of the end 16 of the tube 13 during a procedure to be able to determine the location of the end 16 in the pericardial space.

With reference now to FIG. 5A, details of the multi-lumen tube 12 will now be discussed. The multi-lumen tube 12 includes the proximal end 14 and a second end 40 to which will be connected an end 42 of the tube 13. The tube 12 can have a diameter suitable for its intended purpose. In the case of left atrial appendage closure, the tube can have a maximum diameter of about 5.9-8.6 mm or 18-26 Fr.

The tube 12 comprises a polymer extrusion, for example Pebax®, urethane, nylon, polyethylene, or polypropylene, defining a plurality of separate and distinct lumens. In the illustrated embodiment, the tube 12 has, for example, 5 lumens. A larger or smaller number of lumens can be used depending upon the number of tools to be used in the device 10. In the illustrated embodiment, the tube 12 includes a guidewire lumen 48, a suction lumen 50, an endoscope lumen 52, a grasper lumen 54, and a knot pusher and suture sleeve lumen 56. The lumens 48-56 extend from the end 14 to the end 40.

The tube 13 is also a polymer extrusion, for example Pebax®, urethane, nylon, polyethylene, or polypropylene, defining less lumens than the multi-lumen tube, preferably having one or two lumens. The tube 13 can be a clear or transparent material, and can be employed to create a field of view for a visualization or scoping device. The tube 13 is joined to the end 40 of the tube 12 at juncture 44 (FIG. 2) in a suitable manner, for example using a thermal bond or an adhesive bond. In some embodiments, the tube 13 has a single lumen 66 that extends from the end 42 to the end 16. The space defined by the lumen 66 is large enough to receive portions of the grasping tool 32, the constricting tool 34, and other tools used during the procedure when they are retracted or stowed, as shown in FIG. 3. In embodiments where a guidewire is used, the tube 13 also includes a guidewire lumen that extends from the end 42 to the end 16 and which is aligned with the guidewire lumen 48 of the tube 12 when the tubes 12, 13 are connected.

With respect to the entire tube 11, it will be appreciated that both the multi-lumen tube 12 and the lumen tube 13 may be formed of a single lumen, where various instruments and treatment materials are not compartmentalized into separate and distinct lumens or channels.

When a guidewire is used, the guidewire lumen 48 of the tube 12 and the guidewire lumen in the tube 13 allow the sub-assembly 5 to be inserted over a guidewire, and through an access or introducer sheath when employed, the end of which has previously been positioned adjacent the left atrial appendage. This facilitates positioning of the end 16 of the tube 13 adjacent the left atrial appendage and helps ensure that the proper position of the sub-assembly 5 is maintained. A guidewire also can help maintain and/or regain access to the body lumen or cavity if the device 10 or another instrument is needed to be withdrawn and/or re-introduced. It will be appreciated that guidewires are well known and are commercially available.

The suction lumen 50 allows removal of blood and other fluids and tissue from the pericardial space to improve visibility. Suction can be applied through the lumen 50, or via a suction device that can be introduced through the lumen 50.

The endoscope lumen 52 is used to introduce an endoscope through the sub-assembly 5 to allow visualization of the pericardial space. The endoscope that is used can be a single use, disposable endoscope that is devoid of steering, and can include a lens, vision and light fibers, each of which are conventional in construction. In this embodiment, the endoscope would be discarded after use along with the remainder of the closure sub-assembly 5. The disposable endoscope can be built into the closure sub-assembly 5 so that it is in the optimal position to provide the required direct vision of the left atrial appendage or other internal organs and/or structures. However, the operator will have the ability to unlock the endoscope and reposition it if the procedure requires.

Alternatively, the endoscope can be a commercially available reusable endoscope currently used in the medical field. However, many commercial endoscopes are too large for the direct vision requirements of a left atrial appendage closure device because they contain features, for example steering, excessive light and vision fibers, and working channels, that are unnecessary for the device 10 disclosed herein. Further, the field of view and the working distance of the lens of many commercially available endoscopes may be wrong for use in the left atrial appendage area in the pericardial sac. Further, reusable endoscopes are often damaged either in use or during reprocessing so that they are not available for use when needed.

FIG. 5B shows a schematic illustration of an endoscope 52a extending through the endoscope lumen 52 of multi-lumen tube 12. Like reference numbers as in FIG. 5A are not further described. It will be appreciated that the endoscope 52a is structured and functions as described above so as to be suitable for use with the device.

The grasper lumen 54 and the knot pusher and suture sleeve lumen 56 of the tube 12 open into the lumen 66 (FIG. 3) that is formed in the tube 13. The grasping tool 32 extends through the grasper lumen 54 and into the lumen 66, and the constricting tool 34 extends through the knot pusher and suture sleeve lumen 56 and into the lumen 66.

With reference to FIGS. 1-4, the grasping tool 32 comprises a clamp device 170 formed by two jaw members 172a, 172b that are pivotally connected to each other at pivot 174. A flexible support 176 is connected to the clamp device 170 and extends through the tubes 12, 13 to the actuator 20. The support 176 is used to axially advance the clamp device 170 past the end 16 of the tube 13 from the stowed position shown in FIG. 3 to the extended position shown in FIGS. 1, 2 and 4. The flexible support 176 can bend during use, as shown in FIG. 1. Actuating wires 178 extend through the support 176 and are connected at one end of the jaw members 172a, 172b and at their opposite ends to the actuator 20. The actuating wires 178 are used to open and close the jaw members 172a, 172b for clamping and releasing the appendage 2, by pivoting the jaw members 172a, 172b relative to each other.

The jaw members 172a, 172b each include front teeth and a rear portion 180 formed without teeth to provide an open space between the jaw members. This improves clamping of the appendage 2 by the jaw members, by allowing the appendage tissue to be disposed in the space between the jaw members at the rear, while the front teeth of the jaw members clamp onto the appendage.

The constricting tool 34 can take on a number of configurations. Generally, the tool 34 includes a closure member that is designed to constrict around the left atrial appendage for closing the appendage, and at least one tool to deploy, control, and position the closure member for closing the appendage.

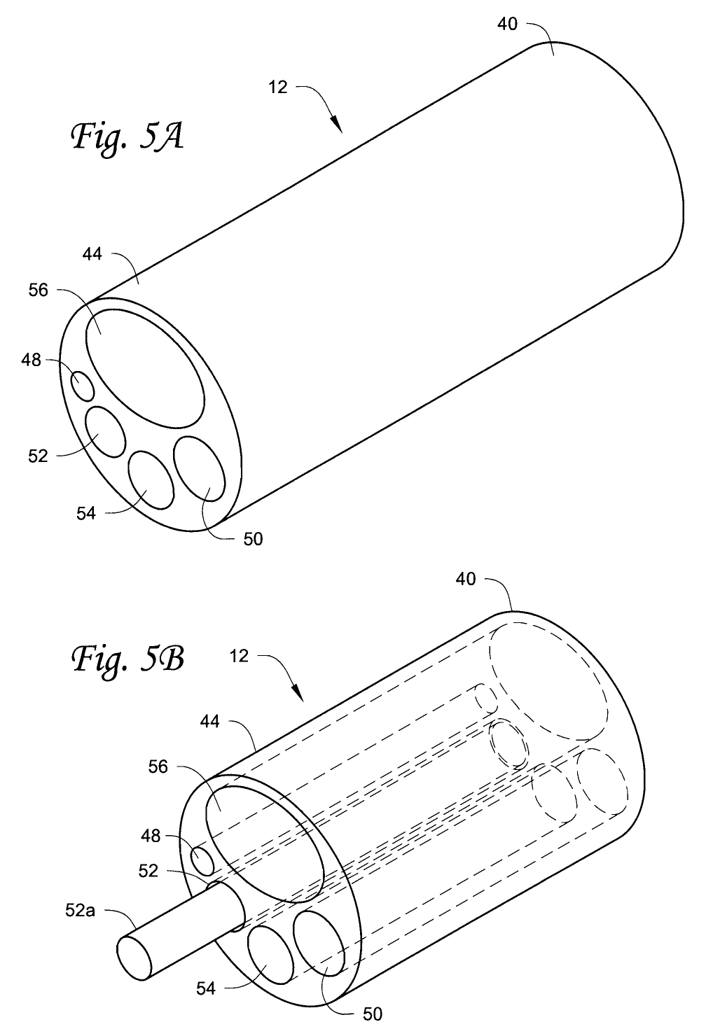

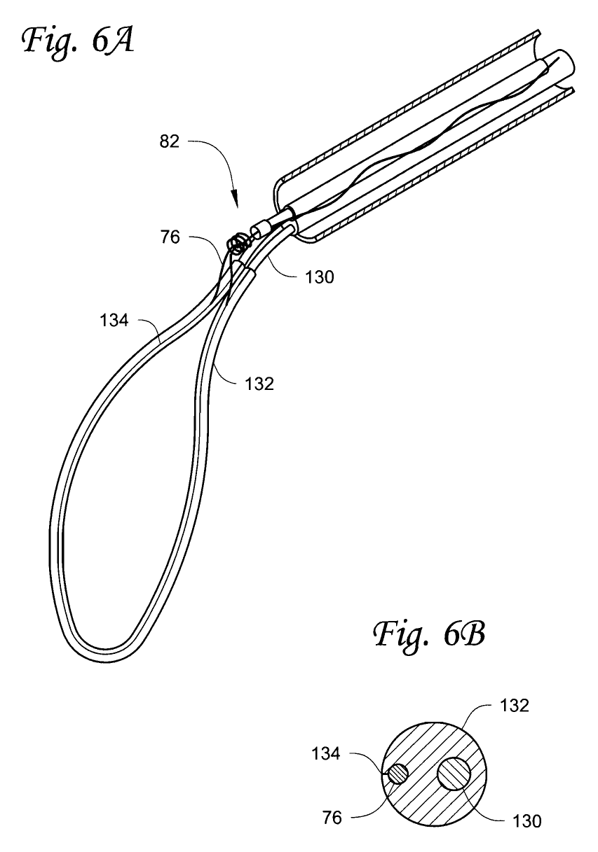

The tool 34 is visible in FIGS. 1-4 and is shown in detail in FIGS. 6A and 6B. The tool 34 includes a support 130 encased in a polymer sleeve 132. In addition, the sleeve 132 substantially encapsulates the closure member, which may be a snare 76 used to close the appendage 2. As shown in FIG. 6B, the sleeve 132 can at least cover or encapsulate the snare 76 substantially around the entire circumference of the snare 76. A slit or thin film 134 is formed in the sleeve 132 through which the snare 76 can be pulled out of the sleeve 132 when the snare 76 is constricted. Generally, the slit or thin film is a line of weakness allowing the snare 76 to be peeled out of the sleeve when the snare is pulled. It will be appreciated that the support 130 may not extend around the entire loop as shown, and may also be a two line feed through a portion of the sleeve from the proximal end. In such a configuration, the support 130 would terminate before being extended through the entire loop of the sleeve 132, such that the length of the sleeve 132 is greater than the length of the support 130.

The snare support 130, which is connected to the actuator 22, for instance, through the mechanism 82 (further described below), and is used to axially advance and retract the constricting tool between the positions shown in FIGS. 3 and 4. The snare support 130 is formed from a suitable shape memory material or super elastic material, for example nitinol or other metal or polymer material which can provide a suitable level of elastic or pseudo-elastic deformation. The snare support 130 expands to generally the shape shown in FIGS. 2 and 4 when extended from the tube 13 in order to expand the snare 76 and maintain the profile of the snare loop. The snare support 130 should expand sufficiently to open the snare 76 sufficiently to ensure a large enough loop so that the snare can fit around the left atrial appendage. The polymer sleeve 132 prevents the snare support from damaging tissue of the patient during use. The sleeve 132 need only encase those portions of the snare support 130 that in use will project past the end 16 of the tube 13.

The snare 76 can be made of any material suitable for encircling and constricting anatomical tissue, and that is biologically compatible with the tissue. For example, the snare 76 can be made of polyester or polypropylene. The snare material can have a diameter of, for example, 0.5 Fr.

The snare 76 includes a pre-tied knot 78, and a mechanism 82 is provided for engaging the knot 78 during tightening or constricting of the snare 76 and cutting the snare 76. The knot 78 can be any suitable knot that allows tightening of the snare 76 by pulling on the suture pull wire 30 that is connected to the snare 76. For example, a knot 78 commonly used in endoscopic surgery, for example a locking slip knot called a Meltzer's knot, can be employed.

The construction of the tool 34 provides a number of advantages. For example, the loop formed by the snare support 130 permits a doctor to approach the appendage at different angles, with the loop and the snare 76 being maintained in their fully expanded condition at all angles of approach.

In addition, when the snare 76 is constricted and pulls out of the sleeve 132, no other material or portion of the snare holding structure gets pinned between the appendage 2 and the snare 76 when the snare is constricted. Such a configuration as disclosed can prevent a portion of the snare holding structure getting pinned in this manner, so that loosening of the constricted snare does not occur for instance when the snare holding structure is retracted. The snare 76 and sleeve 132 construction prevents any material from being pinned between the appendage and the snare, thereby avoiding the possibility of loosening the snare.

It also will be appreciated that the snare 76 and knot 78 may be replaced by a similar material and/or structure used for the support 130. That is, the constricting tool 34 may not include the snare support 130 and sleeve 132 as a separate structure to hold and control the snare 76. Rather, the snare 76 itself may be self-supported and pre-formed as a loop by employing a similar material and/or structure used for the support 130 and/or sleeve 132 (but without the slit 134 since there is no need to peel the snare out of a sleeve) See and compare FIG. 6B. In one embodiment, the snare 76 may be structured as a suitable shape memory material in the form of a loop, such as but not limited to a heat shaped polymer, nitinol, other heat shaped metal. In other embodiments, the snare 76 may be a flexible outer material, such as the material for the flexible polymer sleeve 132, which surrounds an inner material, such as the material for the support 130. See and compare FIG. 6B but without the slit 134. The inner material may be any suitable shape memory material in the form of a loop, such as but not limited to a heat shaped polymer, nitinol, other heat shaped metal. Such a configuration may help to protect the inner material of the snare and may also protect from aggravating the left atrial appendage tissue or other tissue when constricted. For example, where a shape metal material is used, the flexible material may help cushion the snare from contact between any tissue and the inner material.

In operation, the snare 76 would be self-supporting. When the snare 76 is extended from the distal end of the device, the snare 76 would expand and open into a loop structure by the nature of the shape memory material. As described above, the snare may be formed as a knot (like knot 78) that can be tightened or constricted using the pull wire 30. It also will be appreciated that such a modified configuration for the snare may operate with the mechanism 82 described herein. In such a configuration, however, the support 130 and sleeve 132 are not necessary as separate structures since the snare has a built in support and protection structure.

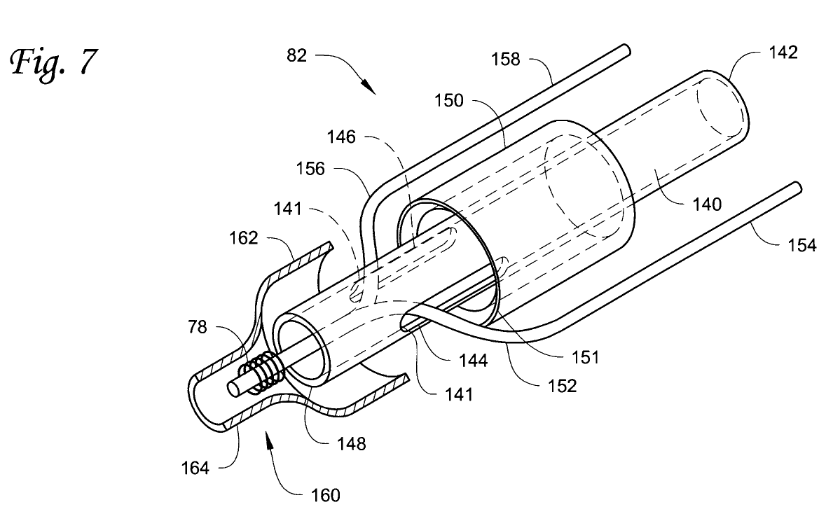

FIG. 7 illustrates further details of the mechanism 82 which is used to advance the snare support 130 and the snare 76 around the left atrial appendage and position the snare at the desired location. The mechanism 82 is connected to the actuator 22 which is used to advance the mechanism 82. The mechanism 82 includes an inner tube 140, and an outer tube 150 surrounding the inner tube 140. The inner tube 140 can be either constructed of relatively small diameter thin wall tubing or a wire material having sufficient diameter. The outer tube 150 can be constructed of either metal or plastic tubing with a metal distal tip as a cutting edge for snare cutting. The outer tube 150 may have a laser cut pattern along the length of the outer tube 150 to make it flexible for increased flexibility and delivery of the device 10.

The outer tube 150 is connected to the snare support 130. The tube 140 is generally hollow, and includes an end 142, a pair of elongated slots 144, 146 that extend from proximate the midpoint of the tube 140 toward a second end 148 of the tube 140. The slots 144, 146 extend through the thickness of the tube 140 to place the interior of the tube 140 in communication with the exterior. The slots 144, 146, have a cutting edge 141 formed on the thickness of the tube 140. The outer tube 150 is sized to cover only a portion of the inner tube 140. For example, in the illustrated embodiment, the tube 150 extends from a point between the end of the slots 144, 146 and the tube end 142 to approximately half the distance of the slots 144, 146. An end 151 of the tube 150 is formed with a sharp cutting edge.

The knot 78 of the snare 76 is disposed adjacent the end 148. One free end 152 of the snare extends into the inner tube 140, out through the slot 144 and along the outside of the outer tube 150 to form a pull end 154. The pull end 154 is designed to tighten or lock the knot 78 when the pull end 154 is pulled. A second free end 156 of the snare extends from inside the inner tube 140, out through the slot 146 and along the outside of the tube 150 to form a pull end 158 which is part of the pull suture 30. The pull suture 30/pull end 158 tightens or constricts the snare around the left atrial appendage once the snare is positioned when the pull suture 30/pull end 158 is pulled.

During constriction and locking, the knot 78 may have a tendency to be pulled to one side or the other which may interfere with constriction and knot locking. Therefore, a closure member support for the knot 78 during these operations may be provided. An example of a closure member support 160 is illustrated in FIG. 7. The closure member support 160 is a generally hollow capsule having a larger diameter end 162 that surrounds the end 148 of the inner tube 140, and a smaller diameter end 164 that surrounds the knot 78. The capsule is fixed onto the inner tube 140.

The mechanism 82 operates as follows. The mechanism 82 is advanced by the actuator 22 which advances and positions the snare around the left atrial appendage. During this time, the inner tube 140 and outer tube 150 maintain their relative positions as shown in FIG. 7. Once the snare is in position, the snare is tightened by pulling on the pull wire 30/pull end 158, which pulls the snare out of the polymer sleeve 132 and constricts the snare about the appendage. Free movement of the free end 156 of the snare is permitted through slot 146. Once the snare is tightened, the knot 78 is tightened or locked by pulling on the pull end 154, with free movement of the free end 152 being permitted through the slot 144. With the snare constricted and the knot tightened, the free ends 152, 156 are then trimmed to length.

Trimming is achieved by retracting the inner tube 140 into the outer tube 150 using the actuator 22. As the tube 140 is retracted into the tube 150, the free ends 152, 156 are pushed to distal ends of the slots 144, 146 having the cutting edges 141 by the outer tube 150. Once the ends of the slots 144, 146 are reached, further retraction of the inner tube 140 causes the cutting edges 141 and end 151 with the cutting edge of the tube 150 to cut the free ends 152, 156. The length of the trimmed ends can be selected by adjusting the length from the end 148 and the cutting edges 141 of the slots 144, 146. Once the snare is cut, the snare support 130 and sleeve 132 can be retracted back into the lumen 66 of the tube 13.

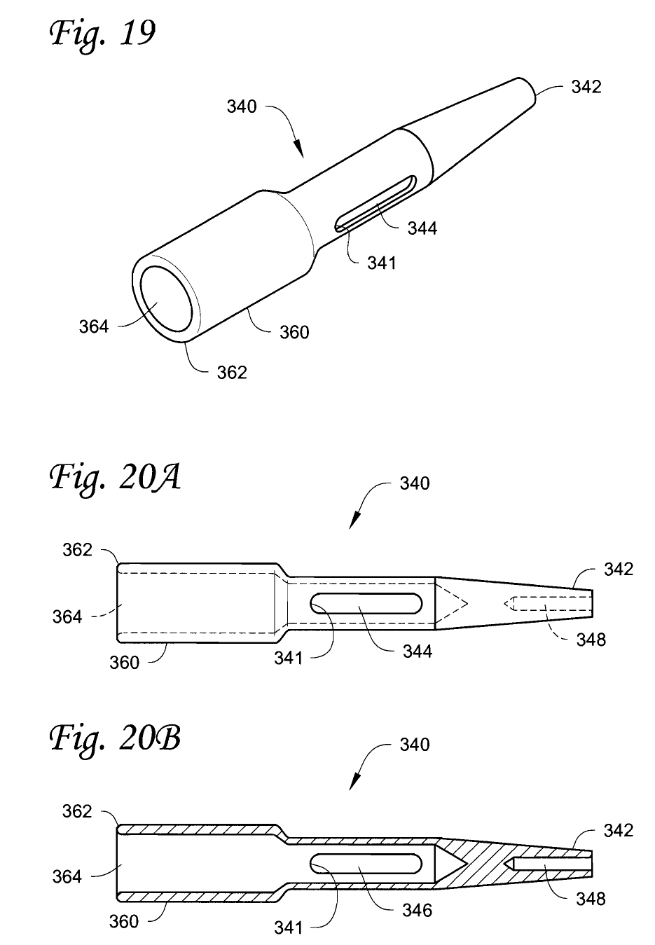

In other embodiments, the inner tube and support means are constructed as a single, unitary, and integral construction. FIGS. 19-20B show an alternative of a tube 340 with a closure support member 360. The tube 340 and the closure member support 360 function similarly as the inner tube 140 and the support means 160 shown in FIG. 7, but include several differences. The tube 340 includes a proximate end 342 and a distal end 362. The tube 340 is generally hollow and includes a pair of elongated slots 344, 346 (best shown in FIG. 20B) each having a cutting edge 341 at the distal radius and that extend from proximate the midpoint of the tube 340 toward the distal end 362. The slots 344, 346 extend through the thickness of the tube 340 to place the interior of the tube 340 in communication with the exterior. As with the inner tube 140, the slots 344, 346 are sharp at the distal ends (at 341) to help in trimming/cutting the snare 76, where the length of the trimmed snare and/or suture ends can be selected by adjusting the length from the end 262 and the distal end of the slots 344, 346.

The closure member support 360 is an integrally formed portion of the tube 340 and is disposed toward the distal end 362. The closure member support 360 acts as a housing for a portion of the closure member, which may be the snare 76. Particularly, the closure member support 360 houses a knot of the closure member (i.e. knot 78 of snare 76). The interior housing size of the closure member support 360 is not particularly limited so long as it is large enough to house the necessary portion of the closure member desired, and so long as it does not conflict with operation of the other tools and components of the device I0. The closure member support 360 is generally hollow and includes an opening 364 at the distal end 362. When the snare 76 and knot are employed, the opening 364 allows for the loop portion of the snare 76 to extend beyond the distal end of the tube 340 (and beyond the overall mechanism 82), such that the snare 76 may be able to operate with the snare support 130 and the closure tool 134 described in FIG. 7 above. The closure member support 360 also is generally hollow toward the middle of the tube 340. That is, the closure member support 360 is in communication with the generally hollow tube 340, so that the snare can be fed through the closure member support 360 and out of the opening 364.

In operation, the closure member support 360 provides a cupping structure to house and protect any knots of the closure member, such as any pre-tied knots of a snare (i.e. knot 78 of snare 76) which may be tightened during operation of the device 10. When the snare 76 is used, such a structure as the closure member support 360 can prevent any material from being pinned or entrapped between the appendage and the snare 76 and/or being pulled inside the knot 78 or snare 76 during the closure operation, which can thereby avoid potential loosening of the snare. Further, the knot 78 when contained under such a construction would not come into contact with other tissue or other inertial structures within the body of a patient.

As shown and described, the tube 340 and closure member support 360 may substitute the inner tube 140 and support means 160 in the mechanism 82 shown in FIG. 7. As with the mechanism 82 of FIG. 7, the outer tube 150 may cover a portion of the tube 340, and where the free ends 152, 156 of the snare 76 may extend into the tube 340, out through the slots 344, 346, and along the outside of the outer tube 150 to form pull ends.

It will be appreciated that the tube 340 and closure member support 360 may be fabricated from various materials including but not limited to stainless steel and plastics. It will be appreciated, however, that such material employed is meant to be non-limiting as long as the material is biocompatible and may be used inside a patient.

As shown in FIGS. 19-20B, the proximate end 342 also may include a thinner profile than other portions of the tube 340. As shown, tube 340 is tapered with a decreasing profile toward the proximate end 342. Such a configuration of the proximate end 342 may allow for easier insertion and fit within an outer tube, for instance the outer tube 150 in FIG. 7. A connecting aperture 348 also may be included at the proximate end 342, where a connective structure such as a pin (not shown) is inserted into the connecting aperture 348 to help hold the tube 340. It will be appreciated that the tube 340, through and as part of the mechanism 82, is connected to an actuator which is used to advance the mechanism, for instance the actuator 22 as described above.

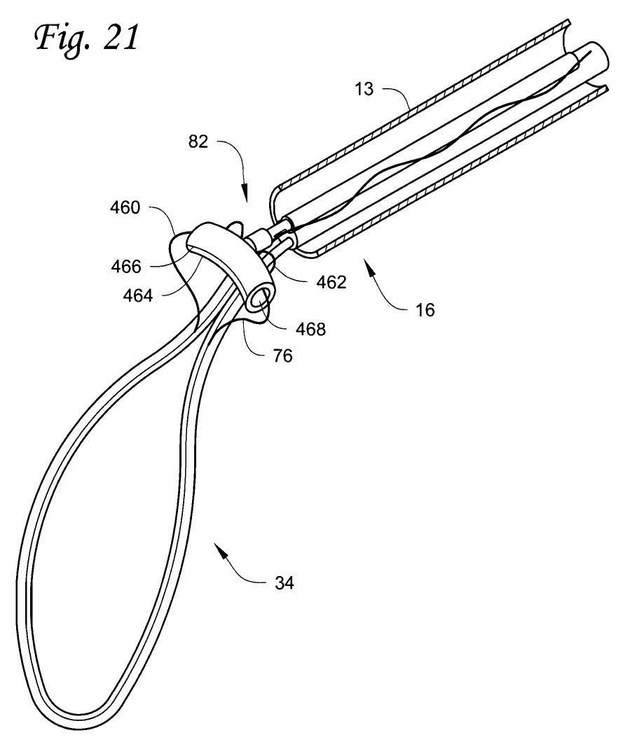

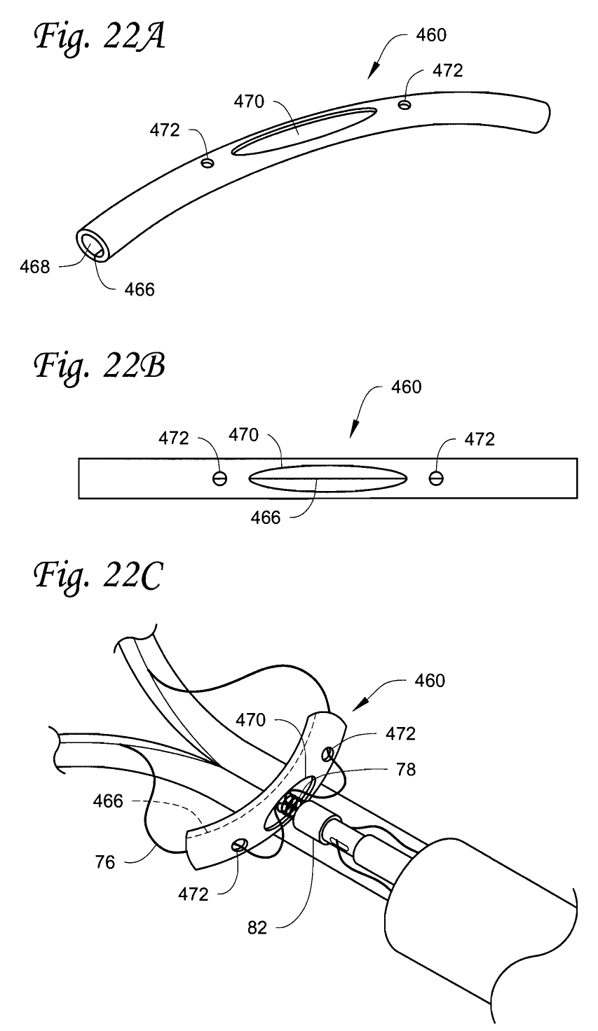

FIGS. 21-22C show yet another alternative for a closure member support. Differently from FIG. 7 and FIGS. 19-20B, a closure member support 460 is disposed distally from the mechanism 82. As with the support means 160 and closure member support 360, the closure member support 460 provides a housing structure to protect any knots of the closure member, such as any pre-tied knots (i.e. knot 78 of snare 76) which may be tightened during operation of the device. Likewise, when the snare 76 and knot 78 are used, the closure member support 460 helps prevent any material from being pinned or entrapped between the appendage and the snare 76 or inside the knot 78 or snare 76 during the closure operation, which can thereby avoid potential loosening of the snare 76 or avoid potential difficulties in tightening the snare 76. Further, the snare knot 78 when contained under such a construction would not come into contact with other tissue or other inertial structures within the body of a patient.

The closure member support 460 is generally a tube that acts as a cover or sleeve to protect a portion of a closure member, for instance the knot 78 of snare 76 or suture. The closure member support 460 includes a side 462 proximate or facing the mechanism 82 and a side 464 distal to or facing away from the mechanism 82. As best shown in FIGS. 22A-B, the side 462 includes an opening 470 where, for example, the knot 78 of snare 76 may enter and be housed within the closure member support 460. Smaller openings 472 allow sides of the loop portion of the snare 76 to be woven therethrough. As shown in FIG. 22C, for example, the sides of the loop diverging away from the knot 78 extend back out of the opening 470, along the outside of the closure member support 460 near the side 462, toward openings 472, and then respectively extend into the openings 472. Side openings 468 are disposed at ends which generally are perpendicular to the longitudinal direction of the device. The sides of the loop diverging away from the knot 78 may exit the side openings 468.

The side 464 distal to or facing away the mechanism 82 includes a slit 466 that is precut into the closure member support 460. The slit 466 provides a line of weakness along a longitudinal profile of the closure member support 460, where portions of the loop of the snare 76 may peel out of the closure member support 460, while providing the cover structure to protect the knot 78. The slit 466 helps for easier removal of the loop of the snare 76 when the snare 76 is to be tightened around the appendage 2.

As shown and described, the closure member support 460 may substitute the support means 160 in the mechanism 82 shown in FIG. 7 and be incorporated distal to the inner and outer tubes 140, 150. The mechanism 82 operates as described above, except with the closure member support 460 disposed at the end to cover and protect the knot 78. In some embodiments, the closure member support 460 may have a dimension (or length from opening 648 to opening 648) sufficient to cover the knot 78 of the snare 76. In other embodiments, the closure member support 460 may have a dimension that is long enough to cover the knot 78 and cover about half or even the entire loop of the snare 76. In such a configuration, portions of the closure member support 460 which may cover portions of the loop of the snare 76 that diverge from the knot 78 would be housed within the sleeve 132 of the constricting tool 34.

The closure member support 460 may be fabricated from various materials such as but not limited to biocompatible polymers and flexible materials. As one example, the closure support member 460 may be fabricated of a polyester material, which may be desirable as the snare sometimes may be a polyester material. It will be appreciated, however, that the particular material used is not limited as long as the material employed is suitable for use inside a patient. It further will be appreciated that the closure member support 460 may either be left behind with the snare or be removed from the patient body with the device upon completion of the procedure.

As described, the closure support member 460 can help prevent tissue from being entrapped in a suture knot. Such a structure as shown and described can help avoid breakage of the snare loop and avoid loosening of the snare loop. Such a structure can also help avoid tissue folding over certain structures of the device tools, where such folding could complicate removal of the device and/or its tools after a procedure.

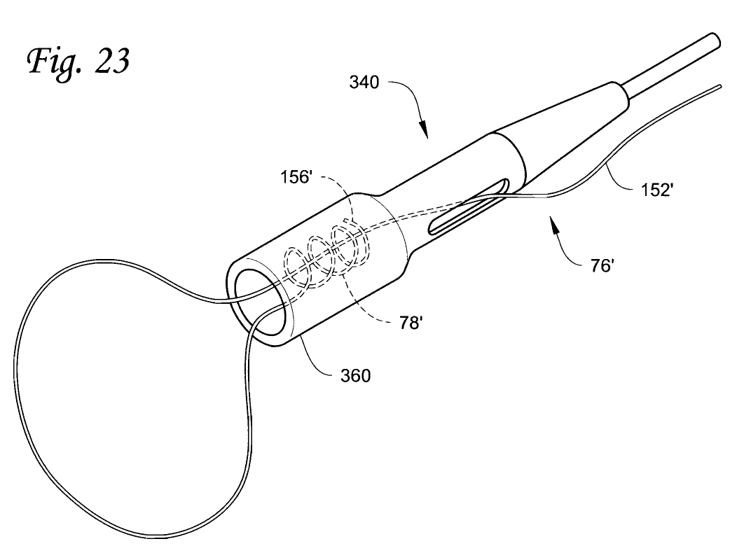

FIG. 23 illustrates another embodiment of a closure member. As shown, a snare 76′ includes a single pull leg configuration, rather than the conventional double leg design shown for example in FIG. 7. The snare 76′ includes a pre-tied knot 78′. As with snare 76, snare 76′ may be engaged with the mechanism 82 during tightening or constricting of the snare 76′ and cutting of the snare 76′. The knot 78′ can be any suitable knot that allows tightening of the snare 76′ by pulling on the suture pull wire connected to the snare 76′. For example, the knot 78′ commonly used in endoscopic surgery, for example, a locking slip knot called a Meltzer's knot can be employed.

Differently from the double leg design, the snare 76′ simply includes a single pull leg at free end 152′ and a free end 156′ that terminates at the knot 78′. Such a configuration eliminates the need for an extended second leg or lock leg (see FIG. 7). As shown, the pull leg at free end 152′ can slip inside the knot 78′ and is similar as the free end 152 or pull leg shown in FIG. 7. The difference is that that the other free end 156′ (or lock leg) is cut shorter and is fixed at the knot 78′ rather than extending back to the actuator. The frictional force between the pull leg at free end 152′ and the knot 78′ is relied upon to keep the snare 76′ tight when it is constricted. Such a configuration avoids relying on mechanically pulling or holding an extended free end (i.e. 156 and 158 of FIG. 7) at the actuator side. Also, as the pull leg at the free end 152′ is pulled to reduce the size of the loop of the snare 76′ over the appendage 2 or other soft tissue, the reaction force from the tissue can further increase the friction force to keep the suture knot tight. It has been found in both bench and animal studies that as long as the loop of the snare is in a good position for closure, the suture knot can stay tight even when another pull leg (i.e. lock leg) is not present to tighten the snare from the other free end.

It will be appreciated that the single pull leg design may be incorporated with any of the support means or closure member supports disclosed herein. As further shown in FIG. 23, the snare 76′ is operable with the tube 340 and closure member 360. As another modification to the tube 340 and closure member 360, however, a single slot may be employed rather than two slots in the double leg snare configuration. A similar single slot modification may be made to the inner tube 140 previously described in FIG. 7. It further will be appreciated that the closure member support 460 can also accommodate the modified snare 76′ and knot 78′ as already described.

Other benefits, among others, that can be enjoyed from the single pull leg configuration include a reduced profile of the device along the entire length and a reduction in procedural steps for operating the closure or constricting procedure as there is no need to pull or lock a second pull leg, since one of the pull legs is eliminated.

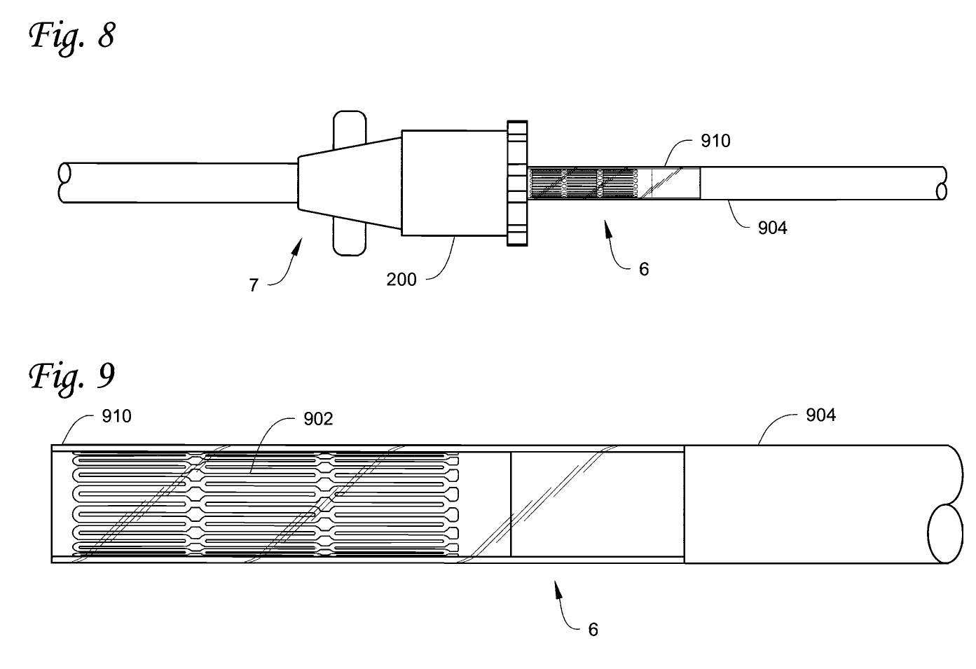

Turning now to the expander sub-assembly 6 and the introducer sheath 7, reference is made to FIG. 8. The introducer sheath 7 is used to create a working channel in a sub-xiphoid procedure for introducing the expander sub-assembly 6 and the closure sub-assembly 5 into the patient. Further details on the introducer sheath 7 can be found in U.S. Patent Application No. 60/938,636, titled Introducer Sheath (attorney docket 20043.18USP1), filed on May 17, 2007, the contents of which are incorporated by reference in their entirety.

The expander sub-assembly 6 is designed to be introduced through the sheath 7 and into the pericardial space for expanding the pericardial space during a closure procedure. Once in position, the expander sub-assembly 6 and the introducer sheath 7 can be locked relative to one another using a locking mechanism 200, the details and operation of which are described in U.S. Patent Application No. 60/938,636, titled Introducer Sheath.

The expander sub-assembly 6 is illustrated in FIGS. 9-13. The expander sub-assembly 6 includes an expanding structure 902 that is a collapsible tool that is self-expanding, collapsible, and constructed of a material utilizing an elastic property. The expander sub-assembly 6 provides key functions in that the expanding structure 902 is retractable and is self-expanding once it is released. In one example, the expanding structure 902 can be configured as a self-expanding shape memory material, which can also be temporarily collapsed when confined. In one embodiment, the expanding structure 902 is a cylindrically hollow part when in an expanded configuration. In this configuration, the expanding structure 902 can allow the constricting tool 34 and the grasping tool 32 to be passed into and through the hollow part of the expanding structure 902, such as when it is expanded.

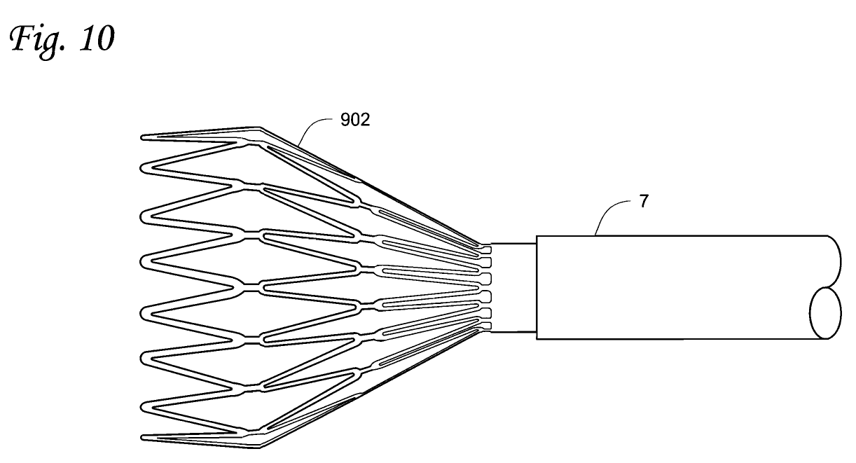

The material of the expanding structure 902 allows it to be collapsed on itself, when it is not deployed. When the expanding structure 902 is not to be deployed, it can be collapsed into a smaller dimension or diameter by being retracted within the elongated body of the introducer sheath 7 (i.e. the shaft structure of the sheath). In operation, the expander sub-assembly 6 can be delivered to a target site such as by extending the expanding structure 902 from the distal end of the elongated body of the introducer sheath as shown in FIGS. 1 and 10, or by retracting the sheath 7 to expose the expanding structure 902. As one example, the expanding structure 902 can be delivered by using a shaft portion 904 that is connected to the end of the expanding structure. The shaft portion 904 is hollow and has an outer diameter that is slightly smaller than the inner diameter of the introducer sheath 7. In this configuration, the shaft portion 904 can be inserted into the sheath and be longitudinally moved within the sheath. As the shaft portion 904 is hollow, the constricting tool 34 and the grasping tool 32 can be passed therethrough.

As shown in FIGS. 8 and 9, the expanding structure 902 is initially held in its collapsed configuration via a loading sheath 910. This permits the expander sub-assembly 6 to be inserted into the introducer sheath 7 as shown in FIG. 8. Once in the sheath 7, the loading sheath 910 is removed or pulled back to free the expanding structure 902. Since the sub-assembly is in the introducer sheath 7, the introducer sheath 7 will hold the expanding structure 902 in its collapsed configuration until the expanding structure 902 is advanced beyond the end of the sheath 7.

The shaft portion 904 can be moved relative to the introducer sheath 7 to extend and retract the expanding structure 902. In the expanded configuration, the expanding structure 902 would be extended past the end of the sheath 7 by pushing it forward relative to the introducer sheath 7, or by pulling the introducer sheath back relative to the expanding structure 902. That is, the introducer sheath can act to cover and uncover the expanding structure 902 based on relative movement of the introducer sheath and expanding structure. In either configuration, the expanding structure 902 can extend from the distal end of the elongated body of the introducer sheath 7 as shown in FIGS. 1 and 10. In the non-expanded configuration, the expanding structure 902 could be collapsed by pulling the expanding structure back inside the introducer sheath 7 through the distal end of the elongated body, or could be collapsed by pushing the introducer sheath over the expanding structure 902 to cover it.

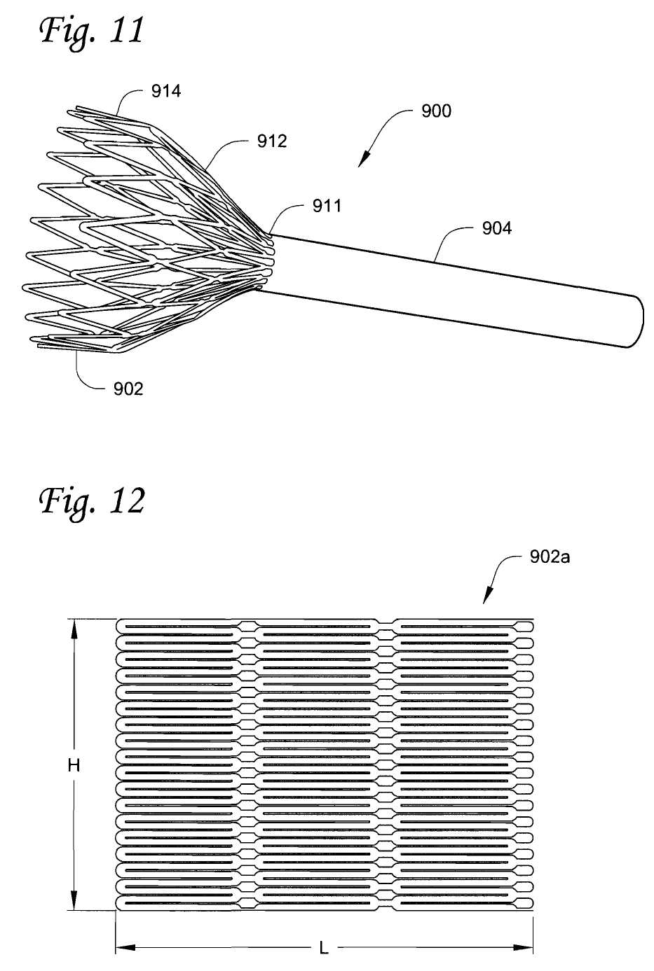

In FIG. 11, when the expanding structure 902 is extended from the sheath, the material of the expanding structure 902 is such that it self-expands to create a working space. That is, due to the expanding structure's propensity to expand when the expanding structure 902 is not contained/retracted inside the access sheath, a space inside a patient can be expanded by the expanding structure.

The expanding structure 902 may be a flexible material with an elastic-like quality, and that includes a self-expanding force that can sufficiently open a working space in the body of a patient. In one embodiment, the expanding structure 902 includes a portion 911 connected to the shaft portion 904, and an outwardly tapering portion 912 that is larger than the outer diameter of the shaft portion 904 and the introducer sheath. The expanding structure 902 also includes a portion 914 distal to the taper portion 912, and that flattens out or becomes generally a uniform circumferential portion. The portion distal to the taper portion further includes tips at the distal end. It will be appreciated that the tips are configured so as not to damage tissue of the body of the patient. In some examples, the tips may be a blunted or rounded structure, such as a paddle-like surface.

As one example, the expanding structure 902 may be a nitinol cage-like structure. It will be appreciated that the expanding structure 902 may be made of materials other than nitinol, for example elastic resins or plastics. It further will be appreciated that the expanding structure 902 may be constructed as a combination of materials, rather than as one material. For example, the expanding portion may be a nitinol or shape memory material, while a proximate portion which connects to the shaft portion may be a stainless steel. It will be appreciated that the materials employed are suitable for use inside the body of a patient.

Likewise, the shaft portion 904 may be sufficiently flexible or have varied flexibility, as necessary or desired, and so as to be suitable for use with the introducer sheath.

FIG. 12 illustrates the expanding device 902 in a flat, unrolled configuration 902a. As described, the expanding portion of the expander sub-assembly 6 may be configured with a cage-like structure. FIG. 12 shows a configuration of the expanding structure 902, which includes a reticulate configuration. It will be appreciated that the expanding structure 902 is not limited to a cage-like configuration, and may not be a reticulated or open structure. Rather, the expanding structure may have a closed outer surface. In one embodiment, the dimensions of structure 902 include a length L of approximately 1.5 inches and a height H of approximately 1.0 inches. The resulting expanding device 902 can have a maximum diameter of, for example, about 40 mm. It will be appreciated that the structure 902 may have varying configurations, and is not limited to the specific configuration shown, as long as the structure 902 can be self-expanding when deployed and collapsible when not in use. It further will be appreciate that the dimensions of the structure 902 may vary as necessary and/or desired.

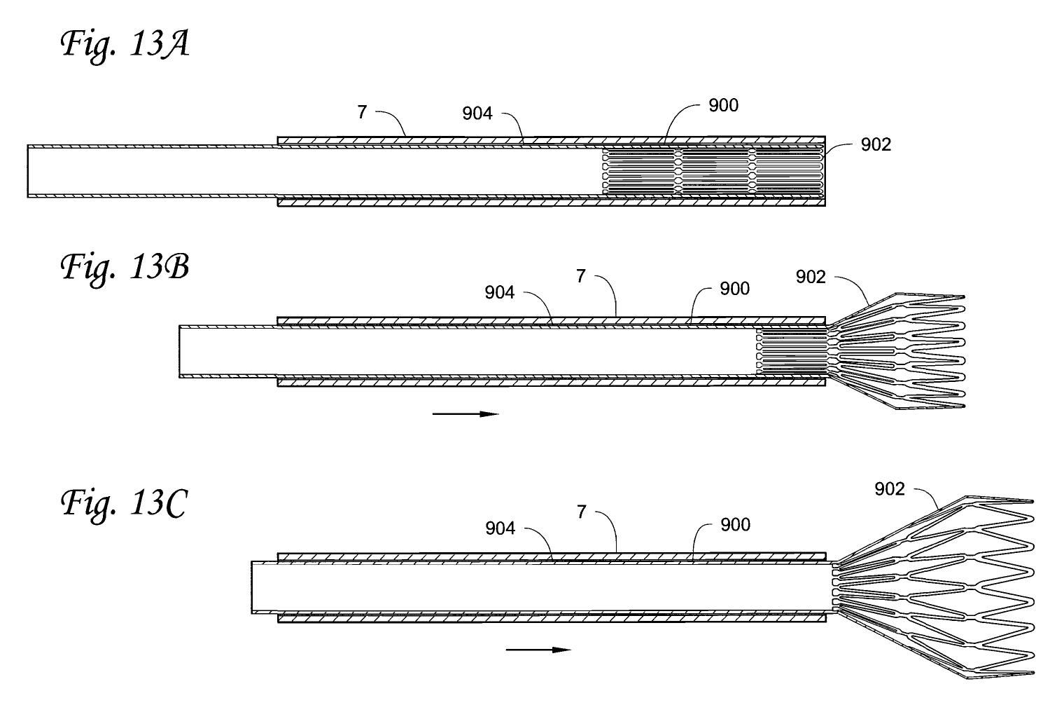

FIGS. 13A-C illustrate side views of the expander sub-assembly 6 in operation with the introducer sheath 7. FIG. 13A shows the sub-assembly 6 in a non-expanded configuration inside the introducer sheath 7. FIG. 13B shows the sub-assembly being advanced axially in the direction of the arrow, with the expanding structure 902 in a partially expanded configuration and partially extended from the sheath 7. FIG. 13C shows the sub-assembly 6 advanced further axially, with the expanding structure 902 in a fully expanded configuration.

When using the device 10 for left atrial appendage closure, the device 10 can be introduced using a sub-xiphoid approach similar to that described in U.S. Pat. No. 6,488,689. In use, once the sheath 7 is in place in the patient, the expander sub-assembly 6 is introduced into the sheath 7. The loading sheath 910 is then removed or pulled back to free the expanding device 902, and the sub-assembly 6 is advanced further axially toward the end of the introducer sheath 7 and the pericardial space. Once it is determined that the end of the sheath 7 is positioned properly, the expander sub-assembly 6 is advanced further until the expanding structure 902 extends past the end of the sheath 7. The expanding structure 902 self-expands to increase the working space. The closure sub-assembly 5 is then introduced through the expander sub-assembly 6 and advanced toward the pericardial space. Once the closure sub-assembly 5 is fully inserted, a locking mechanism can be used to lock the sub-assemblies 5 and 6 together. The locking mechanism can be similar to the locking mechanism 200. The constricting tool 34 and the grasping tool 32 can then be actuated as discussed above to achieve closure of the appendage 2. Once closed, the procedure is reversed to remove the device from the patient.

Alternative embodiments are possible. It will be appreciated that the expander sub-assembly is not limited to the specific structure shown and described, and that other expander constructions and modifications may be employed that are equally or more suitable. For instance, other implementations may include inflatable expanders such as inflatable balloons, or general injection of air into the pericardial space, or any expander structure as may be known in the art that can be suitable for left atrial appendage closure and via a sub-xiphoid, minimally invasive approach.

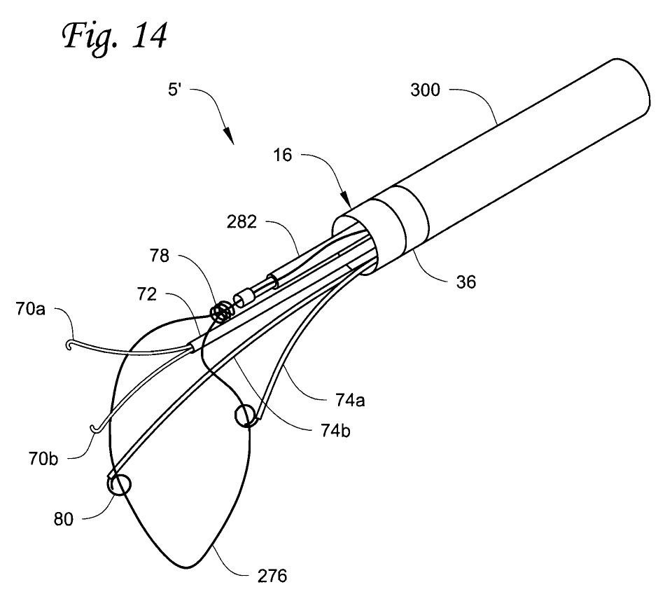

Further, FIG. 14 illustrates an alternative embodiment of a closure sub-assembly 5′ that includes two or more grasping members 70a, 70b that are encased in a sheath 72, with the grasping members and the sheath extending through a multi-lumen tube 300. The grasping members 70a,b can be made of material, for example work hardened stainless steel, such that the grasping members automatically expand outward to the position shown in FIG. 14 when they are extended from the sheath 72. In use, the sheath 72 and the grasping members 70a,b can be extended beyond the distal end 16 by an actuator. The actuator is also used to extend the grasping members 70a,b beyond the sheath 72, as shown in FIG. 14, to permit grasping of the left atrial appendage. The members 70a,b can have a size suitable for performing their grasping function, for example 1.0 Fr.

The closure sub-assembly 5′can also include two or more flexible arms 74a, 74b, a snare 276 with a pre-tied knot 78, a mechanism 80 for releasably connecting the ends of the snare arms 74a,b to the snare 276, and a mechanism 282, similar to the mechanism 82, for engaging the knot 78 during tightening or constricting of the snare 276 and cutting the snare 276. In use, the snare arms 74a,b and mechanism 282 will extend through the multi-lumen tube 300. The snare arms 74a,b can extend to a common attachment point that is ultimately connected to an actuator for actuating the arms 74a,b forwardly, i.e. axially, to advance the snare 276.

The snare arms 74a,b are preferably made of a material that causes the arms 74a,b to automatically expand outward to the position shown in FIG. 14 upon axial advancement of the arm 74a,b beyond the distal end 16. The arms 74a,b should expand sufficiently to open the snare 276 sufficiently to ensure a large enough loop so that the snare can fit around the left atrial appendage. For example, the arms 74a,b can be made of 0.008-0.020 inch diameter nitinol wires that are formed into a gradual lateral curve approximating the shape shown in FIG. 14.

The mechanism 80 for releasably connecting the ends of the snare arms 74a,b to the snare 276 must be able to properly position the snare 276 during positioning of the snare around the left atrial appendage, and must be able to separate from the snare 276 easily and without damaging anatomical tissue or dislocating the snare 276 from around the appendage.

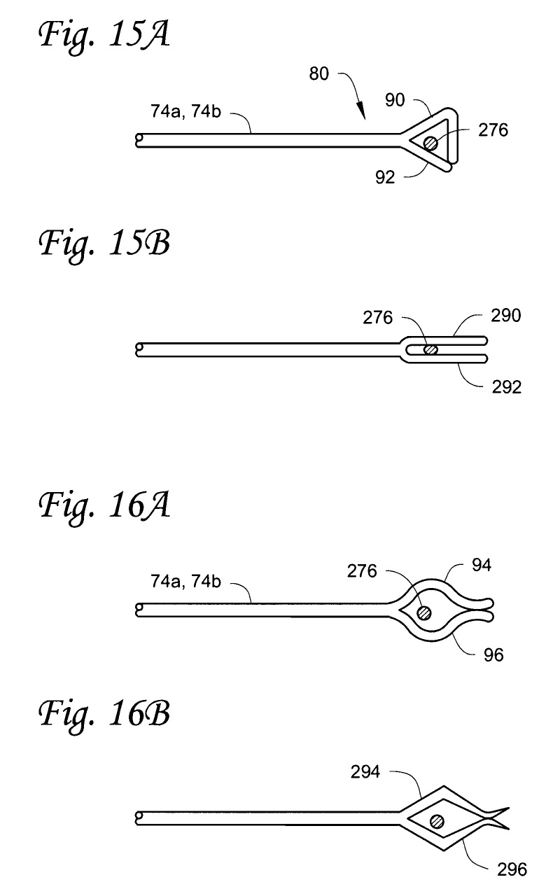

FIGS. 15A and 15B illustrate embodiments of suitable retention/release mechanisms 80. In FIG. 15A, the ends of the snare arms 74a,b terminate in two asymmetric flaps 90, 92 that generally surround the snare 276. The tips of the flaps 90, 92 are positioned adjacent each other, and loosely connected to each other or disconnected entirely. During advancement of the arms 74a,b, the v-shape at the base of the flaps 90, 92 pushes the snare forward. Once the snare is positioned and constricted, and the snare is to be released, the arms 74a,b are pulled backward. As the arms are pulled backward, engagement between the snare 276 and the flaps 90, 92 will cause the flaps to open allowing release of the snare 276.

In FIG. 15B, the snare 276 is held by a friction fit between two flaps 290, 292. Once the snare is positioned and constricted, and the snare is to be released, the arms 74a,b are pulled backward. As the arms are pulled backward, the friction between the snare 276 and the flaps 290, 292 is overcome, open allowing release of the snare 276 through the open end of the flaps 290, 292.

FIGS. 16A-B illustrate more examples of suitable retention/release mechanisms 80. In FIG. 16A, the ends of the snare arms 74a,b terminate in two generally symmetric flaps 94, 96 that generally surround the snare 276. The tips of the flaps 94, 96 are positioned adjacent each other, and loosely connected to each other or disconnected entirely. During advancement of the arms 74a,b, the v-shape at the base of the flaps 94, 96 pushes the snare forward. Once the snare is positioned, constricted, and the snare is to be released, the arms 74a,b are pulled backward. As the arms are pulled backward, engagement between the snare 276 and the flaps 94, 96 will cause the flaps to open, allowing release of the snare 276. FIG. 16B is generally similar, except that the flaps 294, 296 are smaller and create less of a space for the snare 276.

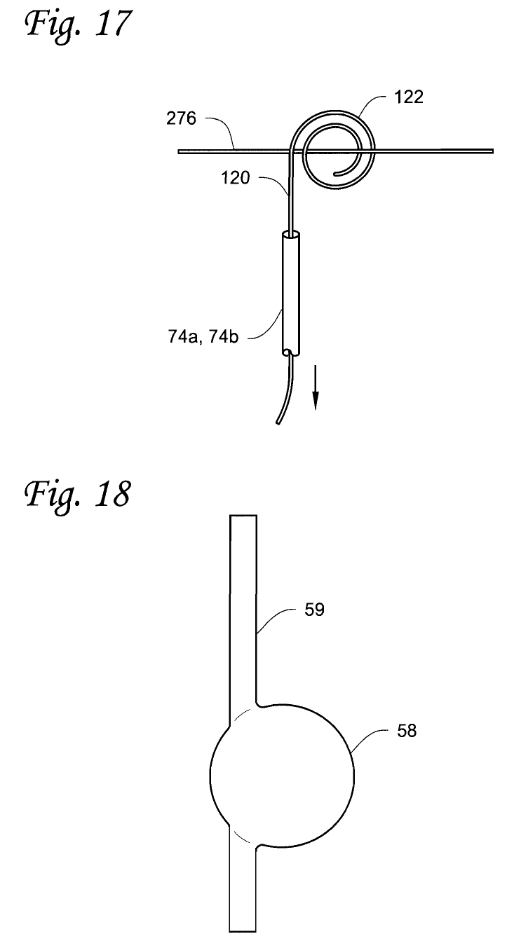

FIG. 17 illustrates another example of a retention/release mechanism 80. In this embodiment, the snare arms 74a,b are hollow tubes and a holding wire 120 made of shape memory material, for example nitinol, extends through each tube. The end of each wire 120 is coiled into a pig-tail 122 around the snare 276. The opposite end of each wire 120 is accessible by the user for pulling and retracting the wire 120 to release the snare. As the wires 120 are retracted, the pig-tail coils 122 unwind, releasing the snare 276.

FIG. 18 illustrates an alternative embodiment of an expander for expanding the pericardial space. FIG. 18 illustrates a balloon 58, shown expanded in FIG. 18, that is bonded on the outside surface of a multi-lumen tube 59 (or a sheath similar to the sheath 7). When inflated, the balloon 58 is asymmetrical in that a larger portion of the balloon, when expanded as shown in FIG. 18, extends to one side of the lumen tube, while a lesser portion of the balloon extends to the other side of the tube so that the balloon is prominent on one side of the tube.

The balloon 58 can be made of, for example, silicone. To facilitate bonding of the balloon 58 to the lumen tube, and to provide a more lubricous surface on the balloon and the tube, a silicone coating can be polymerized to the outer surface of the tube. In addition, the balloon 58 can increase from a diameter of, for example, about 8 mm to, for example, about 40 mm, when expanded.

The balloon 58 can be expanded by, for example, air or a liquid such as saline, introduced into the balloon through a lumen formed in the multi-lumen tube. The lumen can be placed in communication with the balloon 58 via one or more ports (not shown) that extend from the lumen to the exterior of the tube.

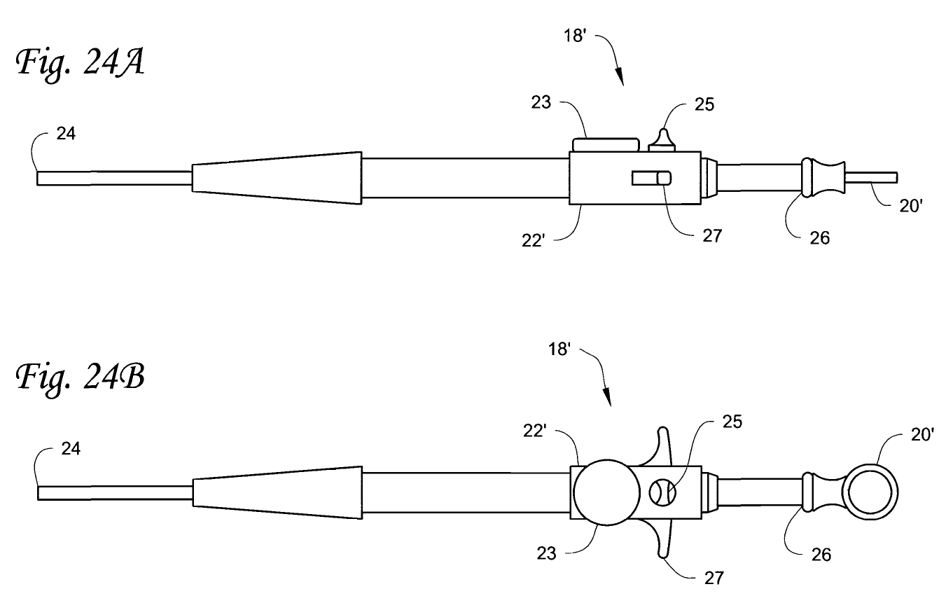

FIGS. 24A-B illustrate an alternative embodiment for actuators that may be used with the closure sub-assembly 5. The actuators 18′ include the following. A pull back or advancing handle 20′ is connected to the grasping tool and for axially controlling the grasping tool. Another pull back or advancing handle 26 is connected to the jaw members and used for opening and closing the jaw members. A snare control actuating mechanism 22′ is connected to the closure tool. The snare control mechanism 22′ includes a rotatable hub 23 for tightening the snare. A knot locker 25 also may be employed as an actuator, for example, when a double leg snare configuration is employed. A trigger 27 is shown which can trim the snare once it has been tightened around the base of the appendage 2. The snare control mechanism 22′ can be a pull back or advancing handle to axially move, deploy, control, and position the closure member.

It will be appreciated that the set of actuators 18, 18′ as shown and described are meant to be non-limiting as a variety of constructions may be employed for deployment, operation, and retraction of the tools of the device which may be equally or more suitable. Such actuator constructions may include but are not limited to other various handles, knobs, and triggers, and may include various ergonomic features as desired and/or suitable, which can be made compatible with the closure sub-assembly 5, as long as the function of the tools and device may be accomplished.

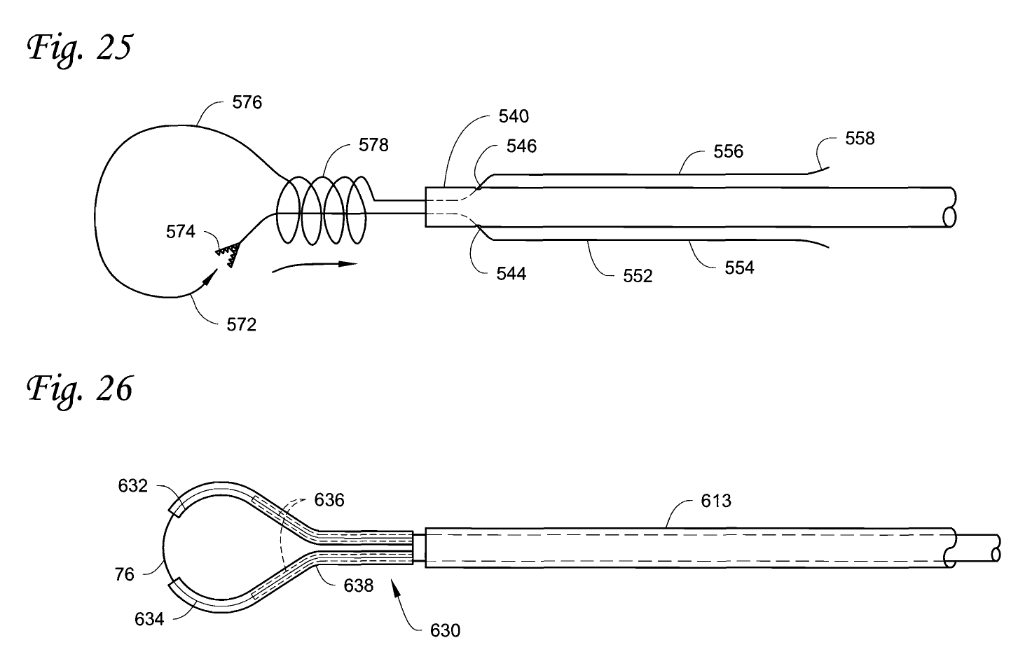

FIGS. 25-35 illustrate additional embodiments, other implementations, and modifications from the general inventive concepts described above. These embodiments are described in sufficient detail to enable those skilled in the art to practice the inventive concepts, and it is to be understood that the following embodiments may be used separately, or be suitably combined with those embodiments already described.

FIG. 25 illustrates another embodiment of a constricting or closure tool. The closure tool generally employs an adjustable snare loop 576 that is delivered using a suture leg 556 and a suture retriever 574. Generally, the suture leg 556 can be advanced underneath or over a target tissue of a subject. The suture retriever 574 is used to retrieve an adjustable free end 572 of the suture leg 556 and engage the adjustable free end 572 to form the loop 576. Then, the suture leg 556 can be pulled by the suture retriever 574 through a pre-tied suture knot 578 to thereby form a two-leg suture snare (with the free end 572). When the suture retriever 574 engages the suture leg 556, the size of the loop 576 can be reduced while the suture retriever 574 is used to further pull the suture leg 556 from the adjustable free end 572. As the size of the loop 576 is reduced, the loop 576 can be tightened to close off a target tissue, such as the left atrial appendage. Once a desired tightness or closure is achieved, the two legs of the suture can be cut using similar principles already described, for example by employing a knot pusher and retractor having a tube and cutting window(s) structure (see 540 described below).

In one embodiment, the suture leg 556 can be fixed or held at free end 558 which is opposite from the adjustable free end 572. It will be appreciated that the free end 558 can also be adjusted or tightened when closing the loop 576, as long as engagement with the suture retriever 572 is not lost or compromised.

The material for the suture leg 556 may be any material already described for a snare material. For example, the suture leg 556 can be any material suitable for encircling and constricting anatomical tissue, and that is biologically compatible with the tissue and for use inside a subject's body. Such materials can include but are not limited to polyester or polypropylene. As shown in FIG. 25, the suture leg 556 in some embodiments may be a shape memory material that is preformed into a partial loop shape but open at the adjustable free end 572 (see distal end of suture leg 556 past the knot and toward the adjustable free end 572). In such a configuration, the suture leg 556 can have a somewhat elastic characteristic so it can be retracted, for example into a lumen tube of a left atrial appendage closure device such as described above, and so it can be extended from a lumen tube and return to its preformed shape. A suitable shape memory material can include, but is not limited to, a heat shaped polymer or metal or nitinol.

In one embodiment, the suture retriever 574 is initially disposed so that it extends inside and through the pre-tied suture knot 578. In such a configuration, the suture leg 556 and suture retriever 574 can be deployed at relatively the same time, since the suture retriever 574 extends through the pre-tied knot 578 and is movably engaged with the pre-tied knot 578 of the suture leg 556. As shown, the suture retriever 574 is slidable through the pre-tied suture knot 578, so that the adjustable free end 572 can be drawn into and through the pre-tied knot 578. It will be appreciated that the suture retriever 574 can also be extended or pushed distally from the pre-tied knot 578, for example when the suture retriever 574 is used to ‘find’ and engage the adjustable free end 572 of the suture leg 556.

The suture retriever 574 is a joining member connected to one end of a pull leg 552. The pull leg 552 can be pulled from an end 552 opposite the end that the joining member is disposed. It will be appreciated that the end 552 extends to a proximate end to be connected with an actuator that allows for the pull leg 552 to be pulled. Such an actuator can be, for example, as shown above for a left atrial appendage closure device.

In some embodiments, the pull leg 552 may be the same material as the suture leg 556, or in other embodiments the pull leg 552 may be a different material from the suture leg 556. The material for the pull leg 552 in some examples can include, but is not limited to, a polyester or polypropylene material, a metal, resin, or polymer material, where the material can be relatively flexible.

The joining member of the suture retriever 574 is constructed and arranged to engage and hold the adjustable free end 572 of the suture leg 556, so that the joining member can form a loop with the suture leg 556 and so that the suture leg 556 can be pulled. As shown, the joining member of the suture retriever 574 is a jaw structure with two jaw members. The jaw structure may be similarly constructed as the two jaw member of clamp device 170 described above and also include a similar actuating wire 178 to open and/or close the jaw structure from the actuator end. It will be appreciated that the joining member is not limited to the jaw structure as shown and may be any suitable structure that can sufficiently engage and hold the adjustable free end 572 of the suture leg 556 so that it can be pulled. As other examples, the joining member may be a clip, grasper, or other suitable mechanical structure or fastener such as a screw, bolt, or rivet that can suitably mate and engage with the adjustable free end 572 of the suture leg 556. In yet other examples, the joining member may include a magnet where the adjustable free end includes a metal material that the magnet can attract and engage. The retriever can also be operated under either direct (for example scope) or indirect (for example under fluoro) visualization techniques.

Similar to the mechanism 82 used with the constricting tool 34 described above, a mechanism is used to deploy the suture leg and suture retriever of the closure tool in extended and retracted positions. As shown, a tube 540 similar to tube 140 can be used to push the suture leg 556 and the suture retriever 574, since the pre-tied knot 578 is disposed outside the opening at the distal end of the tube 540 and it is larger in dimension than the opening. The tube 540 includes cutting windows 544, 546 to cut the suture leg 556 after the adjustable free end 572 has been pulled through the pre-tied knot to a desired position and loop size. To cut the suture leg 556 proximate the adjustable free end 572, the tube 540 can be pulled through an outer tube having an end with a cutting edge such as similarly described above (e.g. outer tube 150 and end 151).

It will be appreciated that the constricting or closure tool of FIG. 25 may be used in conjunction with a lumen tube and may be used alone or in conjunction with the other operating tools and actuators previously described, for example, as part of a left atrial appendage closure device described herein. For instance, the closure tool of FIG. 25 may be suitably used with the multi-lumen tube and its knot pusher suture sleeve lumen 56 and may be suitably used in coordination with the grasping tool described earlier. It should be further recognized that the tool of FIG. 25 may be suitably used with a larger single lumen tool that houses closure and grasper tools described earlier. Likewise, the closure tool may be used in conjunction with suitable visualization components such as a fluoroscopy, ECHO, and endoscope(s) to assist in placement and operation. Even further, the knot 578 of the closure tool of FIG. 25 may be protected by a support or cover, as the knot 578 extends outward from the tube 540. It will be appreciated that any of the supports or covers such as described above may be suitably used with the closure tool of FIG. 25.

As shown in FIG. 25, the configuration also can provide a separately operated suture leg and suture retriever sub-tools. One benefit of the design concept shown is that the suture snare can be looped around a difficult area to reach, while achieving good closure of the snare around a target tissue.

FIG. 26 illustrates another embodiment of a tool to deploy, control, and position a closure member of a constricting tool. As described, a constricting tool can take on a number of configurations. In left atrial appendage closure applications, for example, a constricting tool generally includes a closure member designed to constrict around the left atrial appendage for closing the appendage, and at least one tool to deploy, control, and position the closure member for closing the appendage.

As shown in FIG. 26, a tool to deploy, control, and position a closure member includes a support 630 encased in a polymer sleeve 632. More particularly, FIG. 26 shows an embodiment where the support 630 does not extend around the entire loop. Rather, the support 630 includes a two line feed 636 through a portion of the sleeve 632 from the proximal end. In such a configuration, the support 630 would terminate before being extended through the entire loop of the sleeve 632. That is, the length of the sleeve 632 can be greater than the length of the support 630 at least about the loop portion. In one embodiment, the support 630 includes bend portions 638 that are pre-formed to flare outward when extended from a lumen tube 613, but can be made to collapse so that it can be retracted within the lumen tube 613.

The support 630 is formed from a suitable shape memory material, for example nitinol or other metal or polymer material which can provide a suitable level of elastic deformation. When extended from the tube 613, the support 630 expands to generally the shape shown in FIG. 26 so that a closure member therein can expand and be put into a loop shape (e.g. snare 76). The support 630 should sufficiently expand to open the closure member into a large enough loop so that the closure member can fit around a target tissue, such as the left atrial appendage.

As with the sleeve 132 described above, the sleeve 632 substantially encapsulates a closure member, which may be a snare (e.g. snare 76) used to close the appendage. A slit or thin film 634 also is formed in the sleeve 632 through which the snare can be pulled out of the sleeve 632 when the snare is constricted. It will be appreciated that a slit does not have to be employed in any of the embodiments described herein. Generally, any suitable line of weakness can be employed, such as where a skin of the sleeve 632 is thin or has a “weak” line along the length of the sleeve relative to the other portions of the sleeve 632, and where a closure member can break free of the sleeve 632 when it is pulled. As some examples, the “weak” line can be made from an incomplete cut slit or a thin “skin” along the length of the sleeve 632. For ease of description, the closure member or snare is generally shown, but the details are not specifically shown. The closure member, however, can be similar to the snare as already described (e.g. snare 76), and can include a pre-tied knot and be extended and retracted when used in coordination with for example, a knot pusher such as previously described (see for example FIGS. 6A-B and FIG. 7).

As with sleeve 132 shown in FIG. 6B, the sleeve 632 at least covers the closure member substantially around its entire circumference. As also shown in FIG. 26, the sleeve 632 in some embodiments may not extend around the entire loop shape. Rather, the sleeve 632 may terminate before it covers the entire snare 76.

In other embodiments, it also will be appreciated that the sleeve 632 may not employ a support 630 of shape memory material, and can be formed of a shape memory material itself. In such a configuration, the sleeve 632 can expand to open the closure member or snare into a loop to fit around the target tissue. As shown, only a portion of the sleeve 632 is formed of a shape memory material, so that the sleeve 632 is not entirely composed of a shape memory material (i.e. does not include the entire loop) that encapsulates the closure member or snare.

It will be appreciated that the tool of FIG. 26 may be used alone or in conjunction with the other operating tools and actuators previously described, such as part of a left atrial appendage closure device described herein. For example, the tool of FIG. 26 may be suitably used with the multi-lumen tube and its knot pusher suture sleeve lumen 56 and may be suitably used with the closure and grasping tool described earlier. It should be further recognized that the tool of FIG. 26 may be suitably used with a larger single lumen tool that houses closure and grasper tools described earlier. Likewise, the closure tool may be used in conjunction with suitable visualization components such as a fluoroscopy, ECHO, and endoscope(s) to assist in placement and operation.

Among other benefits, such a support configuration can reduce an outward force of the support such as when the support, sleeve, and snare are retracted, while maintaining a suitable loop profile and pushing ease with the sleeve.

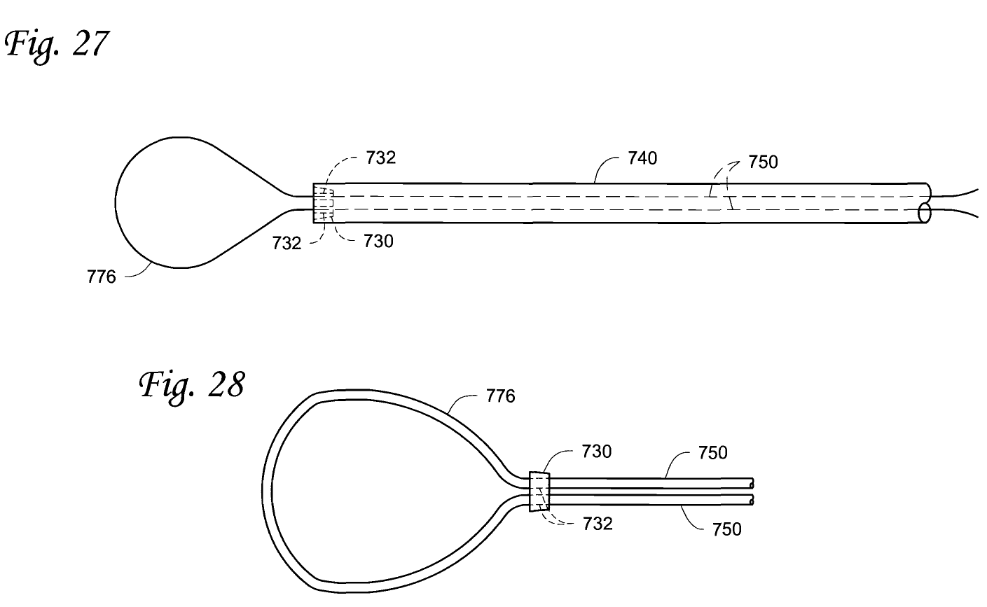

FIGS. 27 and 28 illustrate another embodiment for deploying, controlling, and positioning a closure member, where a pre-tied suture knot is not employed. Generally, a closure member 776 is inserted through holes 732 of a plug 730, where the plug 730 is inserted into an end of a tube 740 which is used as a pusher. The closure member 776 is pushed using the configuration and arrangement of the plug 730, the tube 740, and closure member 776, which is further described below. FIG. 28 shows the plug 730 and the closure member 776 formed as a loop and including suture legs 750.

The closure member includes suture legs 750, so that the closure member 776 can be pulled and tightened around a target tissue. It will be appreciated that the closure member 776 can be a material as already described and that is suitable for encircling and constricting anatomical tissue. The closure member 776 is biologically compatible with the tissue and for use inside a subject's body. In some examples, the closure member 776 (or snare) can be a monofilament suture material having sufficient stiffness such that a suture sleeve and support are not needed. As other examples, the closure member 776 can be formed of a shape memory material as already described. In yet other examples, the closure member 776 may be incorporated and used in conjunction with a support and sleeve structure such as shown and described above in FIGS. 6A and 6B (e.g. support 130, sleeve 132). When such a support and sleeve are employed, the closure member 776 can be peeled out of the sleeve when it is pulled and similar to the operation described above.

The closure member 776 includes legs that are inserted through holes 732 of the plug 730. In one embodiment, the legs of the closure member 776 have an outer dimension that is slightly larger than the size of the holes 732, but where the legs have an outer dimension that allows the closure member 776 to still be pulled through the holes 732 using a sufficient amount of force. For example, the closure member 776 can be made of a softer, flexibly tolerant material that, and the holes 732 of the plug can be made relatively more rigid than the closure member. In such a configuration, when the legs are inserted into the holes 732, the closure member 776 can temporarily reduce its dimension so that its legs snugly fit within the holes 732.

It will be appreciated that the size of the holes 732 can be slightly smaller than the outer dimension of the closure member 776, but where the surface of the holes are flexible to allow some variance in the size of the holes 732 when the closure member 776 is inserted through the holes 732. In such a configuration, a similar effect of a snug fit can be achieved when the closure member 776 is inserted through the plug 730. As shown in FIG. 27, it also will be appreciated that the closure member 776 is still flexible along its length so that it can bend and form the loop.

In operation, the closure member 776 (e.g. snare) can be pushed by the tube 740 and put into position, but also tightened by pulling the suture legs 750 to reduce the size of the loop. FIG. 27 shows an embodiment of an adjustable snare loop without the need of a pre-tied suture knot. As with the tube 140, tube 740 may be extended and retracted from another lumen tube, such as a single and/or multi-lumen tube as described above in a device used for left atrial appendage closure. The loop of the closure member 776 may be secured by joining the suture legs 750, for example at a position distal of the plug 730 and tube 740. In some embodiments, the suture legs may be joined by an adhesive or heated to fuse the suture legs together without using a knot.

In one example where an adhesive is used, the adhesive can be self-contained inside the plug 730. When pressure is applied onto the plug 730 by squeezing the plug 730 with the tube 740, adhesive can be released from pores around the holes 732 to secure the suture to the plug 730. The suture legs 750 and plug 730 can then be cut or trimmed, for example by a cutting tool inserted through the lumen of tube 740, pulled back and removed. The same suture trimming method described above (one or two cutting windows on inner tube) can also be incorporated in this design to cut the suture.

As another alternative for joining the suture legs, a pliers-like or grasper device with hot jaws (not shown) can also be inserted through the lumen of tube 740 to thermally weld the two suture legs 750 together, which can also cut the suture from a location proximal to the welded joint formed by hot jaw. In such an embodiment, the material for the suture can be a thermoplastic polymer or the like which can be thermally welded together and then cut by the hot jaw. It will be appreciated that the hot jaw can be any suitable grasper and can incorporate principles of grasper structures already described.

It will be appreciated that there are a variety of non-limiting ways to secure the closure member to the target tissue. In other embodiments, the plug 730 may be released from the tube 740 so that it remains inside a subject's body. It will be appreciated that the plug is formed of a material that is biologically suitable for use inside the body. The suture legs 750 may be joined, for example at a position that is more proximate from the plug 730 and before the suture legs 750 reach the plug 730. In other examples, an adhesive may be applied within the holes 732 of the plug 730, or the holes 732 of the plug 730 may include a heat activated adhesive to secure the suture legs 750 within the holes 732. In yet another example, the material of the plug 730 inside the holes 732 may be heated to shrink the size of the holes 732 onto the closure member 776. It also will be appreciated that the closure member 776 also may be tightened by mechanically pushing the plug 730 out of the tube 740 and then using any of the above described implementations to secure the closure member 776. It also will be appreciated that the plug 730 and the holes 732 can be designed/made so that the suture legs 750 can only move proximally (e.g. toward the actuator side). Thus, after the suture legs 750 are cut, the plug will not inadvertently dissociate from the suture legs and the suture legs will remain inside the holes 732 of the plug 730.

As another alternative, the plug 730 can be made from a material that can be plastically deformed. For example, the plug 730 can be constructed so that it can be compressed by the tube 740, where the compression force will collapse the holes 732 and restrict the movement of the suture legs 750.

It will be appreciated that the tool of FIGS. 27 and 28 may be used alone or in conjunction with the other operating tools and actuators previously described, such as part of a left atrial appendage closure device described herein. For example, the tool of FIGS. 27-28 may be suitably used with the multi-lumen tube and its knot pusher suture sleeve lumen 56 and may be suitably used with the grasping tool described earlier. It should be further recognized that the tool of FIGS. 27 and 28 may be suitably used with a larger single lumen tool that houses closure and grasper tools described earlier. Likewise, the closure tool may be used in conjunction with suitable visualization components such as a fluoroscopy, ECHO, and endoscope(s) to assist in placement and operation.

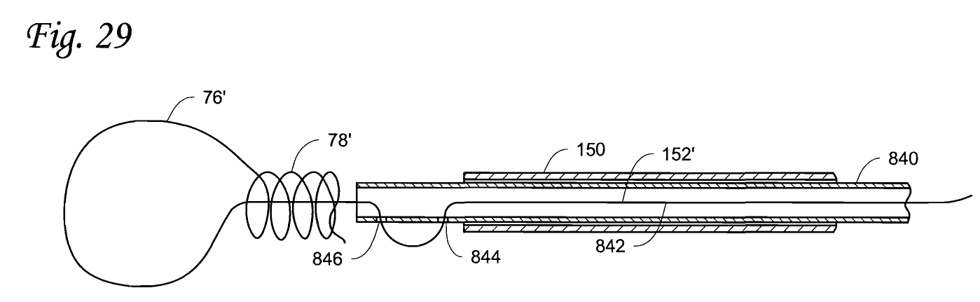

FIG. 29 illustrates an embodiment for deploying and cutting a single suture pull leg 76′ (e.g. also shown in FIG. 23). FIG. 29 shows a tube 840 that can be inserted through outer tube 150. The tube 840 can be used with mechanism 82 as described above, and the tube 840 includes some modifications from inner tubes 140, 340. Generally, the tube 840 is elongated so as to extend substantially through the outer tube 150, and includes a lumen 842 longitudinally extending therethrough. The tube 840 allows for the single suture pull leg at free end 152′ to be loaded through the lumen 842, where the pre-tied knot 78′ resides out of the tube 840 at the distal end. It will be appreciated that any of the closure member supports described above may be employed to protect the pre-tied knot 78′ if desired and/or necessary (e.g. 160, 360, and 460 in FIGS. 7, 22, and 23).

The tube 840 includes at least two cutting windows 844, 846 proximate the distal end. As shown, the cutting windows 844, 846 are generally aligned along the longitudinal direction of the tube 840, where cutting window 844 is upstream from cutting window 846. The cutting windows 844, 846 allow for the single suture pull leg to extend out of the tube 840 by passing through window 844 and extend back into the tube 840 by passing through window 846. The exposed portion of the single suture pull leg provides a cutting region for the suture leg to be cut. As the tube 840 retracts into tube 150, the exposed portion of the single suture pull leg may be cut by the sharp edge of the tube 150 and any sharp edge provided within the windows 844, 846. The single suture pull leg may be cut at window 844 or at both windows 844, 846 depending on the extent the tube 840 is retracted. It will be appreciated that the size of the windows 844, 846 is not meant to be limiting as long as the suture leg can pass through the windows and so that the windows do not disrupt cutting of the suture leg. The spacing of the windows 844, 846 provide the proximity of the cut relative to the pre-tied knot, for example where window 844 is positioned. It further will be appreciated that the spacing of the windows is not meant to be limiting and that various spacing distances between the windows may be employed as desired and/or necessary.

In one embodiment, the tube 840 is made of a hypotube with the lumen 842, where the single suture pull leg 152′ can pass through the windows 844, 846. Among other benefits, such a configuration offers a lower profile design.

It will be appreciated that the embodiment of FIG. 29 may be used alone or in conjunction with the other operating tools and actuators previously described, for example, as part of a left atrial appendage closure device described herein. For instance, the tool of FIG. 29 may be suitably used with a single lumen tube and/or the multi-lumen tube and its knot pusher suture sleeve lumen 56 and may be suitably used in coordination with the grasping tool described earlier. Likewise, the tool may be used in conjunction with suitable visualization components such as a fluoroscopy, ECHO, and endoscope(s) to assist in its and operation.

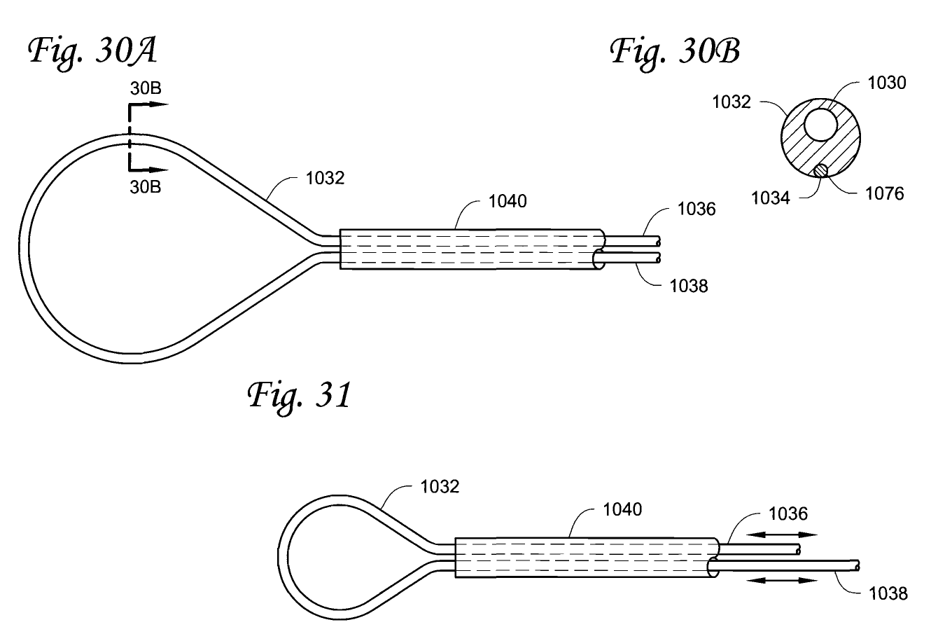

FIGS. 30A-31 illustrate another embodiment of a tool to deploy, control, and position a closure member of a constricting tool. As described, a constricting tool can take on a number of configurations. In left atrial appendage closure applications, for example, a constricting tool generally includes a closure member designed to constrict around the left atrial appendage for closing the appendage, and at least one tool to deploy, control, and position the closure member around the appendage.

As with constricting tool 34 described above, a tool to deploy, control, and position a closure member includes a support 1030 and a closure member 1076 that are encased in a polymer sleeve 1032 (see cross section in FIG. 30B) so as to form a loop. More particularly, FIGS. 30-31 show a modified embodiment where the sleeve 1032, support 1030, and closure member 1076 as an assembly are adjustable prior to release (e.g. peel out) of the closure member 1076.

FIG. 30A shows the loop of the closure member 1076 in an extended position from the distal end of tube 1040. The sleeve 1032 substantially encapsulates the closure member 1076. FIG. 30B shows a cross-section of the sleeve 1032 with two lumens, one lumen for the support 1030 and one lumen for the closure member 1076. The lumen for the closure member 1076 includes a slit 1034 or thin film that allows the closure member 1076 to be released from the sleeve 1032 when the closure member 1076 is tightened. As shown, the slit 1034 or thin film is formed in the sleeve 1032, through which the closure member 1076 (e.g. snare) can be pulled out of the sleeve 1032 when the closure member 1076 is constricted.

In one embodiment, the support 1030 is a shape memory material such as already described and that helps maintain the shape of the loop. For example, the support 1030 can be formed of a nitinol or other metal or polymer material, which can provide a suitable level of elastic deformation. The support 1030 expands to generally the shape shown in FIG. 30A when extended from the tube 1040, and expands the closure member 1076 to maintain the profile of the loop. The snare support 1030 should sufficiently expand to open the closure member 1076 into a large enough loop, so that the closure member 1076 can fit around a target tissue, such as the left atrial appendage.

In other embodiments, it will be appreciated that a separate support 1030 may not be used and that the sleeve 1032 itself can be constructed of a shape memory material to maintain the shape of the loop. In such a configuration, the sleeve 1032 can expand to open the closure member 1076 or snare into a loop to fit around the target tissue. It further will be appreciated that only a portion of the sleeve 1032 can be formed of a shape memory material, so that the sleeve 1032 is not entirely composed of a shape memory material (i.e. does not include the entire loop) that encapsulates the closure member or snare.

FIG. 31 shows the loop drawn down in size. The loop can be drawn into the tube 1040 by pulling one or both legs 1036, 1038 from the proximate end. In such a configuration, the entire loop assembly, including the sleeve 1032, support 1030, and closure member 1076 may be adjusted prior to release and tightening of the closure member 1076 over the targeted tissue (e.g. left atrial appendage). The draw down of the loop assembly serves to first allow for better positioning and placement of the loop around the neck of the left atrial appendage and before tightening or before committing to tighten the closure member 1076. Once the loop is sized to the neck, the closure member 1076 (e.g. snare) can be constricted so it peels out of the lumen of the sleeve 1032 and tightens around the left atrial appendage.

It will be appreciated that the constricting or closure tool of FIGS. 30A-31 may be used in conjunction with a lumen tube and may be used alone or in conjunction with the other operating tools and actuators previously described, such as part of a left atrial appendage closure device described herein. For example, the closure tool of FIGS. 30A-31 may be suitably used with the multi-lumen tube and its knot pusher suture sleeve lumen 56 and may be suitably used with the grasping tool described earlier. It should be further recognized that the tool of FIGS. 30A-31 may be suitably used with a larger single lumen tool that houses closure and grasper tools described earlier. Likewise, the closure tool may be used in conjunction with the tubes 140, 150 for pushing and cutting the closure member 1076. The closure tool also may be used in conjunction with suitable visualization components such as a fluoroscopy, ECHO, and endoscope(s) to assist in placement and operation.

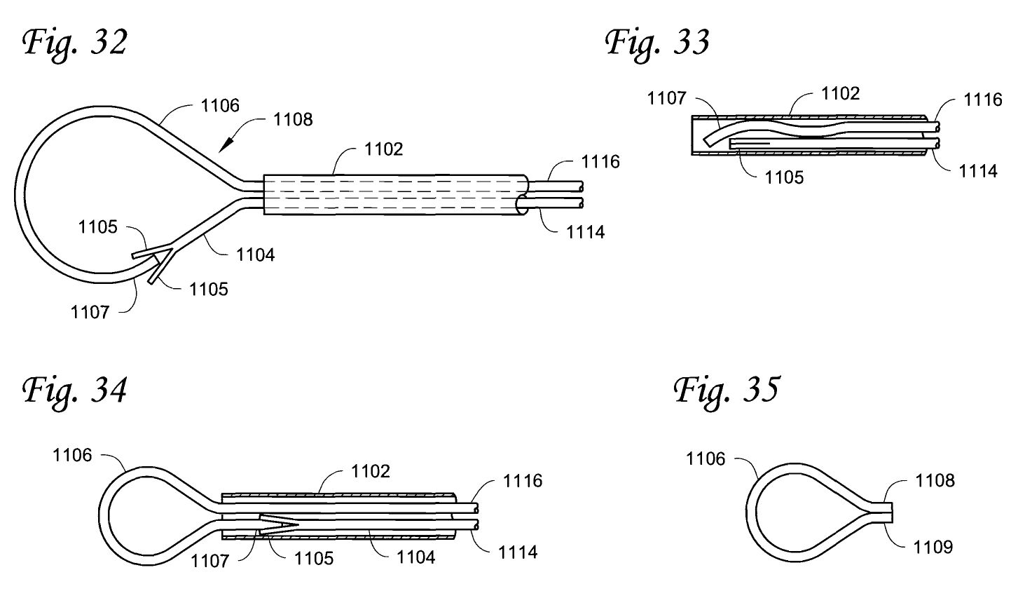

FIGS. 32-35 illustrate another embodiment of a constricting or closure tool. With some similarity to the closure tool shown in FIG. 25, the closure tool of FIGS. 32-35 generally employs an adjustable snare loop that is delivered using a closure member 1106 and a retriever 1104 (see FIG. 33). FIG. 33 shows the closure tool in a retracted position inside lumen tube 1102. FIG. 32 shows the closure tool in an extended position outside the distal end of the lumen tube 1102. Generally, the closure member 1106 can be advanced underneath or over a target tissue of a subject. The retriever 1104 is used to retrieve an adjustable free end 1107 of the closure member 1106 and engage the adjustable free end 1107 to form the loop (see FIG. 32). Once the adjustable free end 1107 is engaged, it is pulled by the retriever 1104 to draw down the size of the loop (see FIG. 34). The size of the loop can be further reduced while the retriever 1104 is used to further pull the adjustable free end 1107 of the closure member 1106. The adjustable free end 1107 can be pulled into the lumen tube 1102. As the size of the loop is reduced, the loop is tightened to close off a target tissue, such as the left atrial appendage. Once a desired tightness or closure is achieved, the closure member can be joined by various suitable mechanisms and implementations as will be described below.

In one embodiment, the closure member 1106 includes a line of material that can be fixed or held at free end 1116 opposite from the adjustable free end 1107. It will be appreciated that the free end 1116 can also made to be adjustable or tightened when closing the loop, as long as engagement with the retriever 1104 is not lost or compromised.

The material for the closure member 1106 may be various materials such as already described. For example, the closure member 1106 is a material suitable for encircling and constricting anatomical tissue, and that is biologically compatible with the tissue and for use inside a subject's body. In some embodiments, the closure member 1106 is a shape memory material that is preformed into a partial loop shape but open at the adjustable free end 1107. In such a configuration, the closure member 1106 can have a somewhat elastic characteristic so it can be retracted, for example into the lumen tube 1102, and so it can be extended from the lumen tube 1102 and return to its preformed shape. A suitable shape memory material can include, but is not limited to, a heat shaped polymer or metal or nitinol. In other embodiments, the closure member 1106 can be a suture material that is relatively flexible (e.g. polyester) and covered or sheathed by a shape memory material. As further examples, the shape memory material can also be constructed inside a braided polymer suture or mono-polymer suture to provide extra support and/or to keep the desired profile of the closure member.

Turning to the retriever 1104, the retriever 1104 includes a joining member connected to a pull leg 1114. The pull leg 1114 can be pulled from an end opposite the end that the joining member is connected. It will be appreciated that the opposite end is connected to an actuator to allow for the pull leg 1114 to be pulled. Examples of actuators have been shown and described above for a left atrial appendage closure device.

In some embodiments, the pull leg 1114 may be the same material as the closure member 1106, or in other embodiments the pull leg 1114 may be a different material from the closure member 1106. As some examples, the material for the pull leg 552 can include, but is not limited to, a polyester or polypropylene material, a metal, resin, or polymer material, where the material can be relatively flexible.

The joining member of the retriever 1104 is constructed and arranged to engage and hold the adjustable free end 1107 of the closure member 1106. In such a configuration, the joining member forms a loop with the closure member 1106 and the closure member 1106 can be pulled by the retriever 1104. As shown, the joining member of the retriever 1104 is a jaw structure with two jaw members. The jaw structure may be similarly constructed as the two jaw member of the clamp device 170 for the grasper tool described above, and can also include a similar actuating wire 178 to open and/or close the jaw structure from the actuator end. It will be appreciated that the joining member is not limited to the jaw structure as shown and may be any suitable structure that can sufficiently engage and hold the adjustable free end 1107 to allow pulling of the closure member 1106. As other examples, the joining member may be a clip, grasper, or other suitable mechanical structure or fastener such as a screw, bolt, or rivet that can suitably mate and engage with the adjustable free end 1107 of the closure member 1106. In yet other examples, the joining member may include a magnet where the adjustable free end includes a metal material that the magnet can attract and engage.

FIG. 33 shows the closure member 1106 and the retriever 1104 pulled or retracted back into the distal end of the lumen tube 1102. The lumen tube 1102 can be constructed similarly as the single lumen tube described above (e.g. lumen tube 13 in FIG. 2) or joined with such single or multi-lumen tubes (e.g. lumen tube 12) as described above. FIG. 32 shows the closure member 1106 and retriever 1104 extended out of the lumen tube 1102. As described above, the closure member 1106 is constructed of a shape memory material, such as a nitinol wire or other shape memory metal or shape memory polymer. When extended from the distal end of the lumen tube 1102, the closure member 1106 in operation can form a portion of a loop. The retriever 1104 can extend from the distal end of the lumen 1102 and be placed in proximity of the adjustable free end 1107 of the closure member 1106. As described, the retriever 1104 includes a joining member attached to a line of material. In the embodiment shown, the joining member is a jaw structure with two jaw members 1105. The joining member is used to engage the adjustable free end 1107 of the closure member 1106 and forms closed loop extending out of the distal end of the lumen 1102.

FIG. 34 shows the loop in a drawn configuration, where the loop is smaller after being drawn in from at least one of the pull legs 1114, 1116.

FIG. 35 shows the loop in a closed or fused position, where the closure member 1106 has a portion 1109 proximate the adjustable free end 1107 that can be joined to a point of weakness 1108. As shown, the closure member is fused through a knotless connection. The closure member 1106 can be joined using a variety of suitable implementations, such as through a clip, grasper, screw, bolt, or rivet or other mechanical connection. In other examples, the closure member 1106 can be joined through heat welding or an adhesive, or if the closure member is a polymer through other methods suitable for joining a polymer material to itself.

It will be appreciated that the constricting or closure tool of FIGS. 32-35 may be used in conjunction with another lumen tube, and may be used alone or in conjunction with the other operating tools and actuators previously described, such as part of a left atrial appendage closure device described herein. For example, the closure tool of FIGS. 32-35 may be suitably used with the multi-lumen tube and its knot pusher suture sleeve lumen 56 and may be suitably used with the grasping tool described earlier. The closure tool herein also can be designed to interface with other tools such as an aspiration tub, thermal welder or clip pusher (for joining the closure member). It should be further recognized that the tool of FIGS. 32-35 may be suitably used with a larger single lumen tool that houses closure and grasper tools described earlier. Likewise, the closure tool may be used in conjunction with suitable visualization components such as a fluoroscopy, ECHO, and endoscope(s) to assist in placement and operation.

As shown in FIGS. 32-35, the configuration also can provide a separately operated closure member and retriever sub-tools. One benefit of the design concept shown is that the suture snare can be looped around a difficult area to reach, while achieving good closure of the snare around a target tissue.

One goal of the medical device and the tools described herein, for example when used for left atrial appendage closure, is to close the appendage at or near its neck so that blood does not move in and out of the appendage. However, it is important not to over tighten the snare so that the appendage is cut by the snare. Therefore, a means of visualizing the opening into the appendage is important. Two known tools that can be used to visualize the movement of blood are Transesophageal Echo (TEE) and Intracardiac Echo (ICE). TEE and ICE allow one to visualize the movement of blood in and out of the appendage in near real time. As the snare is tightened around the appendage, the reduction in size of the appendage neck can be visualized, and the procedure stopped just at the point of no apparent blood flow. In this manner, over tightening of the appendage can be prevented and allow verification that the appendage is closed.

As one general example of performing left atrial appendage closure, an expander is introduced to an area proximate the left atrial appendage through a channel of an introducer sheath. A working space is expanded at the area proximate the left atrial appendage, such as by retracting the introducer sheath to release an expander. A closure sub-assembly is then introduced through the channel of the introducer sheath, where a grasping tool is advanced to grasp the left atrial appendage and grasping the left atrial appendage, and a constricting tool is advanced to close the left atrial appendage including positioning a snare around the left atrial appendage. The left atrial appendage is closed with the snare, and the snare is trimmed. Then, the grasping tool and the constricting tool can be retracted and the working space collapsed to a state before expanding.

The invention may be embodied in other forms without departing from the spirit or novel characteristics thereof. The embodiments disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A constricting tool for minimally invasive access and closure of a left atrial appendage comprising:
a closure member including a generally loop shaped portion configured to constrict around the left atrial appendage when contracted; and
a tool configured to deploy, control, and position the closure member for closing the left atrial appendage, the tool configured to deploy, control, and position the closure member comprising:
a sleeve including a lumen where the sleeve encases the closure member about the circumference of the closure member, and
a line of weakness formed in the sleeve through which the closure member is configured to peel out of the sleeve when the closure member is constricted, the closure member being supported by the sleeve such that the generally loop shaped portion is maintained.

2. The constricting tool of claim 1, wherein the sleeve includes a generally loop shaped portion, where the generally loop shaped portion encases the generally loop shaped portion of the closure member.

3. The constricting tool of claim 1, wherein the tool configured to deploy, control, and position the closure member comprises a loop support disposed within the sleeve, the loop support is formed of a shape memory material that is expandable to maintain the generally loop shaped portion of the closure member and the sleeve.

4. The constricting tool of claim 1, wherein the loop support is formed of a nitinol material.

5. The constricting tool of claim 3, wherein the loop support comprises a two-line feed encased within the sleeve, where the loop support terminates before being extended through the generally loop shaped portion of the sleeve.

6. The constricting tool of claim 5, wherein the two-line feed comprise a bend portion for each line, the bend portions are preformed to bend away from each other and are bendable toward each other.

7. The constricting tool of claim 3, wherein the closure member, sleeve, and loop support are adjustable as an assembly prior to release of the closure member from the sleeve.

8. The constricting tool of claim 1, wherein the closure member is formed of a shape memory material that is expandable to maintain the generally loop shaped portion.

9. The constricting tool of claim 1, wherein the closure member is formed of a nitinol material.

10. A constricting tool for minimally invasive access and closure of a left atrial appendage comprising:
a closure member having a leg with an adjustable free end; and
a retriever having a joining member disposed at one end of a leg, the retriever is configured to engage the adjustable free end of the closure member, the closure member and retriever forming a generally loop shaped portion when the closure member and retriever are engaged, and are configured to constrict the left atrial appendage when contracted.

11. The constricting tool of claim 10, wherein the closure member is a flexible suture leg having a pre-tied knot, the retriever is disposed through the pre-tied knot and is movable with respect to the pre-tied knot, the retriever is configured to draw the adjustable free end through the pre-tied knot when the retriever and the closure member are engaged, and thereby reduce the generally loop shaped portion.

12. The constricting tool of claim 11, wherein the closure member and retriever are configured to be deployed at the same time.

13. The constricting tool of claim 10, wherein the joining member of the retriever comprises two jaw members pivotally connected to each other at a pivot.

14. The constricting tool of claim 10, wherein the closure member is a partial loop shape that is preformed of a shape memory material.

15. The constricting tool of claim 10, wherein the closure member is a nitinol material.

16. The constricting tool of claim 10, wherein the closure member is configured to be joined to itself, the closure member including a point of weakness, such that a portion proximate the adjustable free end is joined to the point of weakness when the closure member has been constricted around the left atrial appendage.

17. The constricting tool of claim 10, wherein the closure member is joined through a knotless connection.

18. The constricting tool of claim 10, wherein at least one of the leg of the closure member and the leg of the retriever is a pull leg.

19. The constricting tool of claim 10, wherein the leg of the closure member is fixed at an end opposite the adjustable free end, and the leg of the retriever is a pull leg.

20. The constricting tool of claim 10, further comprising an imaging and visualization tool.
